(19) United States
(12) Patent Application Publication
(10) Pub. No.: US 2009/0024333 A1
(43) Pub. Date: Jan. 22, 2009

(54) Methods and systems relating to mitochondrial DNA phenotypes

(75) Inventors: Roderick A. Hyde, Redmond, WA (US); Muriel Y. Ishikawa, Livermore, CA (US); Eric C. Leuthardt, St. Louis, MO (US); Dennis J. Rivet, St. Louis, MO (US); Lowell L. Wood, JR., Bellevue, WA (US)

Correspondence Address: SEARETE LLC;CLARENCE T. TEGREENE, 1756 - 114TH AVE., S.E., SUITE 110, BELLEVUE, WA, 98004, US

(73) Assignee: Searete LLC, a limited liability corporation of the State of Delaware

(21) Appl. No.: 11/900,050

(22) Filed: Sep. 7, 2007

Related U.S. Application Data
(63) Continuation-in-part of application No. 11/880,454, filed on Jul. 19, 2007.
Application No. 11/880,454 is a continuation-in-part of application No. 11/880,453, filed on Jul. 19, 2007.
Application No. 11/880,453 is a continuation-in-part of application No. 11/900,051, filed on Sep. 7, 2007.

(51) Int. Cl. G01N 33/48

(52) U.S. Cl. 702/20

(57) ABSTRACT

In one aspect, a system includes, but is not limited to, at least one computer program for use with at least one computer system and wherein the computer program includes a plurality of instructions, including but not limited to, one or more instructions for determining at least one correlation between at least one mitochondrial DNA-influencing event and at least one aspect of mitochondrial DNA phenotype information regarding at least one individual.

### CROSS-REFERENCE TO RELATED APPLICATIONS

The present application is related to and claims the benefit of the earliest available effective filing date(s) from the following listed application(s) (the “Related Applications”) (e.g., claims earliest available priority dates for other than provisional patent applications or claims benefits under 35 USC § 119(e) for provisional patent applications, for any and all parent, grandparent, great-grandparent, etc. applications of the Related Application(s)).

### RELATED APPLICATIONS

For purposes of the USPTO extra-statutory requirements, the present application constitutes a continuation-in-part of U.S. patent application Ser. No. 11/880,454, entitled METHODS AND SYSTEMS RELATING TO. MITOCHONDRIAL DNA INFORMATION, naming Roderick A. Hyde, Muriel Y. Ishikawa, Eric C. Leuthardt, Dennis J. Rivet, and Lowell L. Wood, Jr. as inventors, filed 19 Jul., 2007, which is currently co-pending, or is an application of which a currently co-pending application is entitled to the benefit of the filing date.

For purposes of the USPTO extra-statutory requirements, the present application constitutes a continuation-in-part of U.S. patent application Ser. No. 11/880,453, entitled METHODS AND SYSTEMS RELATING TO MITOCHONDRIAL DNA INFORMATION, naming Roderick A. Hyde, Muriel Y. Ishikawa, Eric C. Leuthardt, Dennis J. Rivet, and Lowell L. Wood, Jr. as inventors, filed 19 Jul., 2007, which is currently co-pending, or is an application of which a currently co-pending application is entitled to the benefit of the filing date.

For purposes of the USPTO extra-statutory requirements, the present application constitutes a continuation-in-part of U.S. patent application Ser. No. ______, entitled METHODS AND SYSTEMS RELATING TO MITOCHONDRIAL DNA PHENOTYPES, naming Roderick A. Hyde, Muriel Y. Ishikawa, Eric C. Leuthardt, Dennis J. Rivet, and Lowell L. Wood, Jr. as inventors, filed 7 Sep., 2007, which is currently co-pending, or is an application of which a currently co-pending application is entitled to the benefit of the filing date.

The United States Patent Office (USPTO) has published a notice to the effect that the USPTO's computer programs require that patent applicants reference both a serial number and indicate whether an application is a continuation or continuation-in-part. Stephen G. Kunin, Benefit of Prior-Filed Application, USPTO Official Gazette Mar. 18, 2003, available at http://www.uspto.gov/web/offices/com/sol/og/2003/week11/patbene.htm. The present Applicant Entity (hereinafter “Applicant”) has provided above a specific reference to the application(s) from which priority is being claimed as recited by statute. Applicant understands that the statute is unambiguous in its specific reference language and does not require either a serial number or any characterization, such as “continuation” or “continuation-in-part,” for claiming priority to U.S. patent applications. Notwithstanding the foregoing, Applicant understands that the USPTO's computer programs have certain data entry requirements, and hence Applicant is designating the present application as a continuation-in-part of its parent applications as set forth above, but expressly points out that such designations are not to be construed in any way as any type of commentary or admission as to whether or not the present application contains any new matter in addition to the matter of its parent application(s).

All subject matter of the Related Applications and of any and all parent, grandparent, great-grandparent, etc. applications of the Related Applications is incorporated herein by reference to the extent such subject matter is not inconsistent herewith.

### SUMMARY

In one aspect, a method includes, but is not limited to, determining a similarity or a dissimilarity between at least one aspect of mitochondrial DNA phenotype information regarding at least one first individual and at least one aspect of mitochondrial DNA phenotype information regarding at least one second individual, wherein the at least one second individual had been influenced by at least one mitochondrial DNA-influencing event. In addition to the foregoing, other method aspects are described in the claims, drawings, and text forming a part of the present disclosure.

In one or more various aspects, related systems include but are not limited to circuitry or programming for effecting the herein-referenced method aspects; the circuitry or programming can be virtually any combination of hardware, software, or firmware configured to effect the herein-referenced method aspects depending upon the design choices of the system designer.

In one aspect, a system includes, but is not limited to, at least one computer program for use with at least one computer system and wherein the computer program includes a plurality of instructions, including but not limited to, one or more instructions for determining at least one correlation between at least one mitochondrial DNA-influencing event and at least one aspect of mitochondrial DNA phenotype information regarding at least one individual. In one aspect, a system includes, but is not limited to, at least one computer program for use with at least one computer system and wherein the computer program includes a plurality of instructions, including but not limited to, one or more instructions for determining a similarity or a dissimilarity between at least one aspect of mitochondrial DNA phenotype information regarding at least one first individual and at least one aspect of mitochondrial DNA phenotype information regarding at least one second individual, wherein the at least one second individual had been influenced by at least one mitochondrial DNA-influencing event. In one aspect, a system includes, but is not limited to, at least one computer program for use with at least one computer system and wherein the computer program includes a plurality of instructions, including but not limited to, one or more instructions for determining one or more correlations between at least one aspect of mitochondrial DNA phenotype information obtained regarding at least one first individual and information regarding at least one mitochondrial DNA-influencing event in relation to the at least one first individual, and one or more instructions for applying at least one of the one or more correlations to at least one aspect of mitochondrial DNA phenotype information regarding at least one second individual. In one aspect, a system includes, but is not limited to, at least one computer program for use with at least one computer system and wherein the computer program includes a plurality of instructions, including but not limited to, one or more instructions for determining one or more correlations between at least one aspect of mitochondrial DNA phenotype information obtained regarding at least one first individual and information regarding at least one medical therapy in relation to the at least one first individual, and one or more instructions for applying at least one of the one or more correlations to at least one aspect of mitochondrial DNA phenotype information regarding at least one second individual. In one aspect, a system includes, but is not limited to, at least one computer program for use with at least one computer system and wherein the computer program includes a plurality of instructions, including but not limited to, one or more instructions for determining one or more correlations between at least one aspect of mitochondrial DNA phenotype information obtained regarding at least one first individual and information regarding at least one environmental event in relation to the at least one first individual, and one or more instructions for applying at least one of the one or more correlations to at least one aspect of mitochondrial DNA phenotype information regarding at least one second individual. In addition to the foregoing, other system aspects are described in the claims, drawings, and text forming a part of the present disclosure.

The foregoing summary is illustrative only and is not intended to be in any way limiting. In addition to the illustrative aspects, embodiments, and features described above, further aspects, embodiments, and features will become apparent by reference to the drawings and the following detailed description.

### BRIEF DESCRIPTION OF THE FIGURES

FIG. 1 illustrates some aspects of a system that may serve as an illustrative environment for subject matter technologies.

FIG. 2 depicts aspects of a system such as the one illustrated in FIG. 1.

FIG. 3 shows aspects of a system such as the one illustrated in FIG. 1.

FIG. 4 illustrates aspects of a system such as the one illustrated in FIG. 1.

FIG. 5 illustrates some aspects of a system that may serve as an illustrative environment for subject matter technologies.

FIG. 6 depicts aspects of a system such as the one illustrated in FIG. 5.

FIG. 7 shows aspects of a system such as the one illustrated in FIG. 5.

FIG. 8 illustrates aspects of a system such as the one illustrated in FIG. 5.

FIG. 9 shows some aspects of a system that may serve as an illustrative environment for subject matter technologies.

FIG. 10 depicts aspects of a system such as the one illustrated in FIG. 9.

FIG. 11 shows aspects of a system such as the one illustrated in FIG. 9.

FIG. 12 illustrates some aspects of a system that may serve as an illustrative environment for subject matter technologies.

FIG. 13 depicts aspects of a system such as the one illustrated in FIG. 12.

FIG. 14 illustrates some aspects of a system that may serve as an illustrative environment for subject matter technologies.

FIG. 15 shows aspects of a system such as the one illustrated in FIG. 14.

FIG. 16 depicts aspects of a system such as the one illustrated in FIG. 14.

FIG. 17 shows a logic flowchart of a process.

FIG. 18 illustrates a logic flowchart of a process, such as the one shown in FIG. 17.

FIG. 19 depicts a logic flowchart of a process, such as the one shown in FIG. 17.

FIG. 20 shows a logic flowchart of a process, such as the one depicted in FIG. 17.

FIG. 21 shows a logic flowchart of a process.

FIG. 22 illustrates a logic flowchart of a process, such as the one shown in FIG. 21.

FIG. 23 depicts a logic flowchart of a process, such as the one shown in FIG. 21.

FIG. 24 shows a logic flowchart of a process, such as the one depicted in FIG. 21.

FIG. 25 shows a logic flowchart of a process.

FIG. 26 depicts a logic flowchart of a process, such as the one shown in FIG. 25.

FIG. 27 shows a logic flowchart of a process.

FIG. 28 illustrates a logic flowchart of a process, such as the one shown in FIG. 27.

FIG. 29 shows a logic flowchart of a process.

FIG. 30 depicts a logic flowchart of a process, such as the one shown in FIG. 29.

### DETAILED DESCRIPTION

In the following detailed description, reference is made to the accompanying drawings, which form a part hereof. In the drawings, similar symbols typically identify similar components, unless context dictates otherwise. The illustrative embodiments described in the detailed description, drawings, and claims are not meant to be limiting. Other embodiments may be utilized, and other changes may be made, without departing from the spirit or scope of the subject matter presented here.

This document uses formal outline headings for clarity of presentation. However, it is to be understood that the outline headings are for presentation purposes, and that different types of subject matter may be discussed throughout the application (e.g., device(s)/structure(s) may be described under process(es)/operations heading(s) or process(es)/operations may be discussed under structure(s)/process(es) headings; or descriptions of single topics may span two or more topic headings). Hence, the use of the formal outline headings is not intended to be in any way limiting.

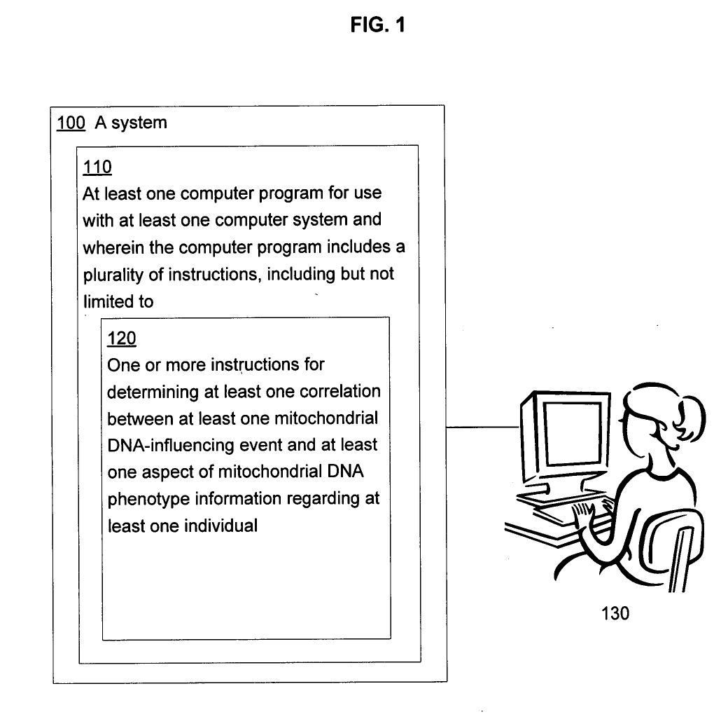

With reference to the figures, and with reference now to FIG. 1, depicted is one aspect of a system that may serve as an illustrative environment of or for subject matter technologies, for example, at least one computer program for use with at least one computer system and wherein the computer program includes a plurality of instructions, including but not limited to, one or more instructions for determining at least one correlation between at least one mitochondrial DNA-influencing event and at least one aspect of mitochondrial DNA phenotype information regarding at least one individual. Accordingly, first described herein are certain specific example systems of FIG. 1; thereafter, there is illustrated certain specific example structures and processes. Those having skill in the art will appreciate that the specific structures and processes described herein are intended as merely illustrative of their more general counterparts.

### A. Structure(s) and or System(s)

Continuing to refer to FIG. 1, depicted is a partial view of a system that may serve as an illustrative environment of, or for, subject matter technologies. One or more users 130 may use a system 100 including at least one computer program 110 for use with at least one computer system, wherein the at least one computer program 110 includes a plurality of instructions. One or more users 130 may include, for example, one or more administrators, medical personnel, pharmacists, geneticists, researchers or technicians. Although a single user is shown in FIG. 1, in some embodiments at least one group of users or at least one series of users may interact with the system. In some embodiments, the one or more users 130 may include a computer system, artificial intelligence system (AI) or other circuitry. The at least one computer program 110 may include one or more instructions for determining at least one correlation between at least one mitochondrial DNA-influencing event and at least one aspect of mitochondrial DNA phenotype information regarding at least one individual.

A correlation may be established by, for example, statistical methods or by a general relationship between the data sets. In some embodiments, the at least one correlation may include at least one statistical correlation. In various aspects, at least one statistical correlation may include, for example, at least one linear correlation, at least one nonlinear correlation, functional dependency or other mathematical relationship. At least one statistical correlation may or may not be associated with some type of causality, real or implied, proven or unproven. At least one statistical correlation may or may not be associated with some type of medical event such as, for example, illness, allergic reaction, bleeding, stroke, one or more side effects, or death.

At least one aspect of mitochondrial DNA phenotype information as described herein may be provided by one or more of a number of sources. Mitochondrial DNA phenotype information, for example, may be provided by one or more array based system, such as the GeneChip® Human Mitochondrial Resequencing Array 2.0, sold by Affymetrix, with corporate headquarters in Santa Clara Calif. See, e.g., the GeneChip® & Human Mitochondrial Resequencing Array 2.0 Data Sheet, which is herein incorporated by reference. Mitochondrial DNA phenotype information may, for example, be provided by a hybridization-based system, such as the Signet™ Mitochondrial DNA Screening System, sold by Marlingen Biosciences Inc., with corporate headquarters in Ijamsville Md. See, e.g., the Signet™ Mitochondrial DNA Screening System brochure, which is herein incorporated by reference. Mitochondrial DNA phenotype information may, for example, be provided by one or more high performance liquid chromatography (HPLC) techniques such as described by Bayat et al., Mitochondrial mutation detection using enhanced multiplex denaturing high-performance liquid chromatography, International Journal of Immunogenetics 32: 199-205 (2005), which is herein incorporated by reference. In some embodiments, mitochondrial DNA phenotype information may be provided by more than one technique or method. See, for example, U.S. Pat. No. 6,967,016 to van Gemen et al., titled “Method of determining therapeutic activity or possible side effects of a medicament,” which is herein incorporated by reference.

As used herein, a “mitochondrial DNA-influencing event” includes an event that influences the mitochondrial DNA phenotype of at least one cell within at least one individual. A mitochondrial DNA-influencing event may, but need not, influence every cell in an individual's body, or influence every cell in an organ, region of the body, or tissue. A mitochondrial DNA-influencing event may influence the sequence of mitochondrial DNA in at least one cell, or it may influence the relative proportion of mitochondrial DNA variants present in one or more cells. For example, a mitochondrial DNA-influencing event may influence the relative proportions of heteroplasmic mitochondrial DNA variants. A mitochondrial DNA-influencing event may be part of a medical therapy, for example a mitochondrial DNA-influencing event may include one or more medications, or one or more treatments. A mitochondrial DNA-influencing event may include at least one medical therapy including one or more energy-based procedures, for example radiation therapy, UV therapy, thermal therapy or ultrasound therapy. A mitochondrial DNA-influencing event may be noticed at the time of the event, or it may not be noticed by the affected individual or other individuals. A mitochondrial DNA-influencing event may include one or more environmental events, such as but not limited to, exposure of an individual to radiation, one or more pollutants, or one or more toxins. A mitochondrial DNA-influencing event may happen in a short time interval such as minutes or hours or it may happen over a long time interval such as weeks, months or years. A mitochondrial DNA-influencing event may include multiple smaller events, such as multiple small exposures that in the aggregate influence the mitochondrial DNA of at least one cell within at least one individual. A mitochondrial DNA-influencing event may include the personal activities of an individual, including an individual knowingly exposing himself to, for example, toxins. As an example, a mitochondrial DNA-influencing event may include the choice of an individual to expose himself to cigarette smoke, or the choice of an individual to work in an environment with a high level of asbestos. As an example, a mitochondrial DNA-influencing event may include the choice of an individual to be in a location where toxins are present, such as in a location where arsenic levels are very high in the drinking water.

As used herein, “mitochondrial DNA phenotype information” includes mitochondrial DNA phenotype information regarding at least one phenotype of at least one cell. For example, mitochondrial DNA phenotype information includes information regarding linear DNA sequence such as polymorphisms or mutations. See, for example, Ruiz-Pesini et al., An enhanced MITOMAP with a global mtDNA mutational phylogeny, Nucleic Acids Research 35: D823-D828 (2007), which is herein incorporated by reference. For example, mitochondrial DNA phenotype information includes information regarding the class, group or homology of one or more mitochondrial DNA sequence regions with at least one known mitochondrial DNA class(es), group(s), or sequence(s). See, e.g. Wallace, Mitochondrial DNA sequence variation in human evolution and disease, PNAS USA 91: 8739-8746 (1994) and Khrapko et al., Mitochondrial mutational spectra in human cells and tissues, PNAS USA 94: 13798-13802 (1997), which are herein incorporated by reference. Mitochondrial DNA phenotype information may include temporal information, spatial information, biochemical information, or metabolic information. Mitochondrial DNA phenotype information may be stable over time in multiple assays or it may alter over time, for example as cells are renewed, refreshed or replaced. Mitochondrial DNA phenotype information may change over time in multiple assays as cells progress through the cell cycle, including through cellular division. The mitochondrial DNA phenotype information relating to different cells may be different at a single time point, even when the cells originate from a single individual, including from a single organ or tissue.

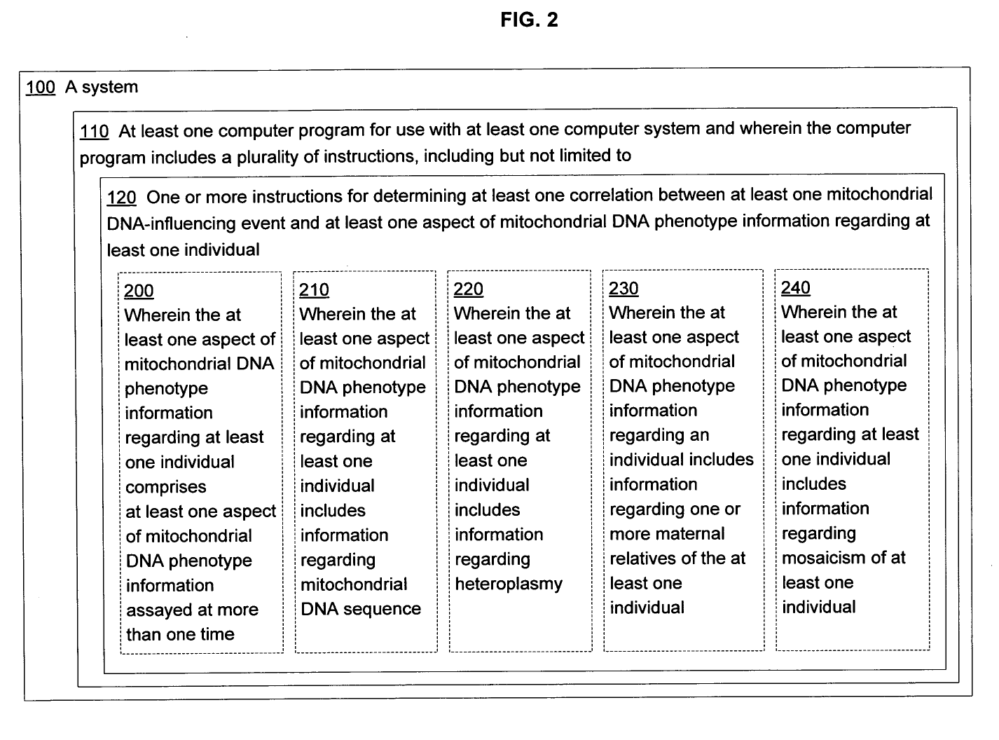

FIG. 2 depicts alternate embodiments of the system of FIG. 1. In some embodiments, the one or more instructions for determining at least one correlation 120 may include at least one of instructions 200, 210, 220, 230 or 240. In some embodiments, the one or more instructions for determining at least one correlation 120 may include embodiments wherein the at least one aspect of mitochondrial DNA phenotype information regarding at least one individual comprises at least one aspect of mitochondrial DNA phenotype information assayed at more than one time 200. For example, aspects of mitochondrial DNA phenotype information may be assayed on multiple occasions or set intervals, such as weekly or monthly. For example, aspects of mitochondrial DNA phenotype information may be assayed in multiple assays carried out in parallel or in series, and the information from the assays combined into a system. For example, at least one aspect of mitochondrial DNA phenotype information may be assayed at irregular intervals, such as when an individual enters into a medical treatment or consults medical personnel. In some embodiments, the one or more instructions for determining at least one correlation 120 may include embodiments wherein the at least one aspect of mitochondrial DNA phenotype information regarding at least one individual includes information regarding mitochondrial DNA sequence 210. For example, information regarding mitochondrial DNA sequence may include information regarding linear DNA sequence such as polymorphisms or mutations. Information regarding mitochondrial DNA sequence may include, for example, information regarding the class, group or homology of one or more mitochondrial DNA sequence regions with at least one known mitochondrial DNA class(es), group(s), or sequence(s). In some embodiments, the one or more instructions for determining at least one correlation 120 may include embodiments wherein the at least one aspect of mitochondrial DNA phenotype information regarding at least one individual includes information regarding heteroplasmy 220. Information regarding heteroplasmy, for example, may include information regarding heteroplasmy between tissue types or within tissue types, and may include information regarding heteroplasmy of an individual or multiple individuals. See, for example, Simonetti et al., Accumulation of deletions in human mitochondrial DNA during normal aging: analysis by quantitative PCR, Biochimica et Biophysica Acta 1180: 113-122 (1992), and Liu et al., Mutations in mitochondrial DNA accumulate differentially in three different human tissues during ageing, Nucleic Acids Research 26: 1268-1275 (1998) which are herein incorporated by reference. In some embodiments, the one or more instructions for determining at least one correlation 120 may include embodiments wherein the at least one aspect of mitochondrial DNA phenotype information regarding at least one individual includes information regarding one or more maternal relatives of the at least one individual 230. For example, information regarding one or more maternal relatives of the at least one individual may include information regarding the individual's biological mother, maternal grandmother, maternal aunts and uncles, or maternal cousins. In some embodiments, the one or more instructions for determining at least one correlation 120 may include embodiments wherein the at least one aspect of mitochondrial DNA phenotype information regarding at least one individual includes information regarding mosaicism of at least one individual 240. The term “mosaicism,” as used herein, denotes, for example, situations where two or more cellular subtypes arise during the lifespan of an organism, situations where two or more cellular subtypes originate with the first cell of an organism and situations where the origin of the cellular subtypes is unclear. The term “mosaicism,” as used herein, may include somatic mosaicism, gonadal mosaicism, or chimerism. For example, information regarding mosaicism of at least one individual may include information regarding mosaicism of two or more tissue types, or mosaicism within at least one tissue type. For example, information regarding mosaicism of at least one individual may include information such as the presence or absence of mosaicism, the location of mosaicism, the tissue or tissues involved in the mosaicism, or the proportion of various subtypes of cells in mosaic tissue. For more information regarding somatic mosaicism, see Youssoufian and Pyeritz, Mechanisms and consequences of somatic mosaicism in humans, Nature Reviews Genetics, 3: 748-758 (2002), which is herein incorporated by reference.

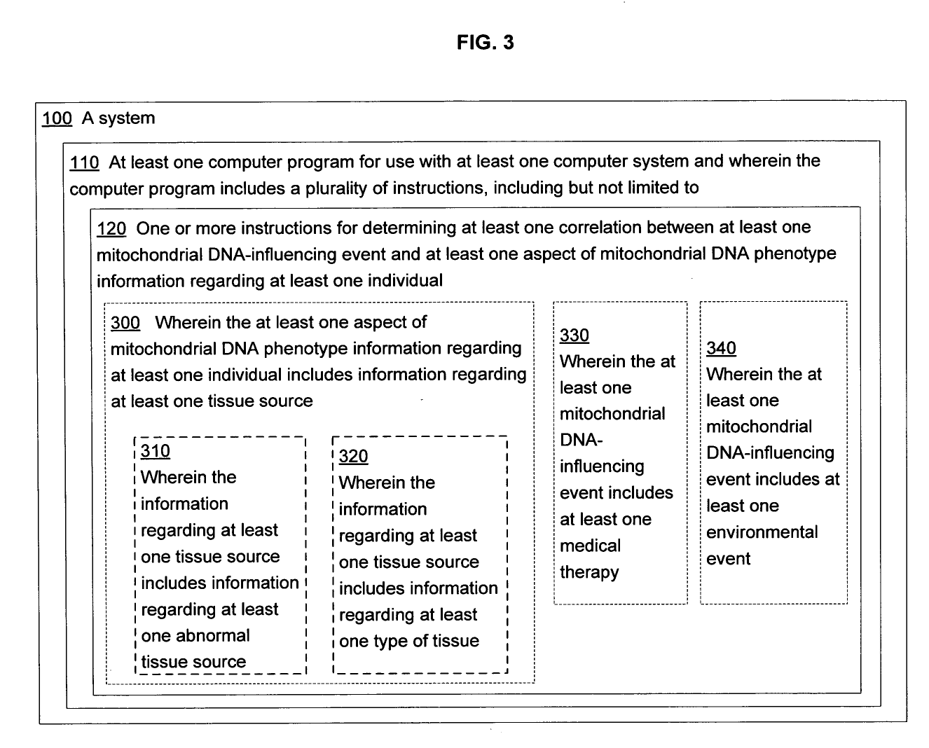

FIG. 3 depicts alternate embodiments of the system of FIG. 1. In some embodiments, the one or more instructions for determining at least one correlation 120 may include embodiments wherein the at least one aspect of mitochondrial DNA phenotype information regarding at least one individual includes information regarding at least one tissue source 300. For example, the information regarding at least one tissue source may include information regarding the origin, storage, pathology, pathological subtype, or handling of the tissue. For example, the information regarding at least one tissue source may include information regarding at least one physical, spatial or relative anatomic source. The information regarding at least one individual includes information regarding at least one tissue source 300 may include information regarding at least one abnormal tissue source 310. For example, the information regarding at least one abnormal tissue source may include information regarding a neoplastic source, a displastic source, a diseased source, an infectious source or a cancerous source. The information regarding at least one individual includes information regarding at least one tissue source 300 may include information regarding at least one type of tissue 320. For example, information regarding at least one type of tissue may include information regarding at least one clinical diagnosis, at least one pathology report, or at least one surgical report. For example, the information regarding at least one type of tissue may include the origin tissue type, the handling of the tissue, or one or more treatments to the tissue. In some embodiments, the information regarding at least one type of tissue may include cellular developmental stage, lineage, or status. For example, the information regarding at least one type of tissue may include information regarding at least one physical, spatial or relative anatomic source. See, e.g., Kujoth et al., The role of mitochondrial DNA mutations in mammalian aging, PLOS Genetics, 3: e24 (2007), which is herein incorporated by reference. In some embodiments, the one or more instructions for determining at least one correlation 120 may include embodiments wherein the at least one mitochondrial DNA-influencing event includes at least one medical therapy 330. For example, a mitochondrial DNA-influencing event may include a medical therapy including one or more medications, or one or more treatments. A mitochondrial DNA-influencing event may include at least one medical therapy including one or more energy-based procedures, for example radiation therapy, UV therapy, thermal therapy, phototherapy, or ultrasound therapy. For example, a mitochondrial DNA-influencing event may include at least one medical therapy with at least one therapeutic agent, wherein the at least one therapeutic agent may for example, include at least one drug, biologic, biological material, formulation, pharmaceutical, nutraceutical, dietary supplement, vitamin or compound. A therapeutic agent may be administered, for example, in a variety of ways including oral, topical, iv, ip, sc, intranasal, and inhalation. In some embodiments, the one or more instructions for determining at least one correlation 120 may include embodiments wherein the at least one mitochondrial DNA-influencing event includes at least one environmental event 340. For example, an environmental event may include exposure of an individual to radiation, one or more pollutants, or one or more toxins. For example, an environmental event may include exposure of an individual to excessive levels of cigarette smoke. For example, an environmental event may include exposure of an individual to excessive levels of toxins such as asbestos, or arsenic. In some embodiments, an environmental event may include some element of activity by an individual, such as when an individual goes to or stays in a location with exposures such as those described herein.

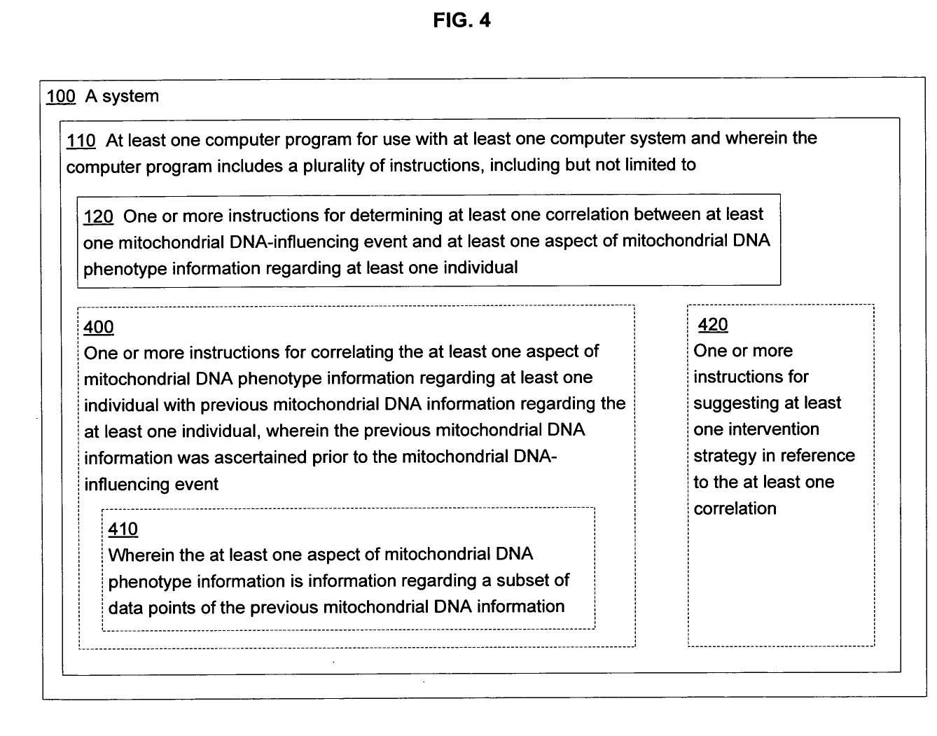

FIG. 4 depicts further aspects of a system such as that shown in FIG. 1. The computer program 110 includes a plurality of instructions and may include one or more instructions for correlating the at least one aspect of mitochondrial DNA phenotype information regarding at least one individual with previous mitochondrial DNA information regarding the at least one individual, wherein the previous mitochondrial DNA information was ascertained prior to the mitochondrial DNA-influencing event 400. In some embodiments, one or more instructions for correlating 400 may include instructions wherein the at least one aspect of mitochondrial DNA phenotype information is information regarding a subset of data points of the previous mitochondrial DNA information 410. For example, an initial assay or group of assays may yield information regarding a large number of data points, while subsequent smaller assays or groups of assays may yield information regarding a smaller group, or subset of data points. For example, an initial assay may yield a large number of data points that are included in the system, while information from further assays that is incorporated into a system is limited to a subset of data points. Also in reference to FIG. 4, the computer program 110 includes a plurality of instructions and may include one or more instructions for suggesting at least one intervention strategy for the at least one first individual in reference to the at least one correlation 420. For example, one or more instructions for suggesting at least one intervention strategy may include one or more instructions for suggesting a course of medication, or a medical treatment. For example, one or more instructions for suggesting at least one intervention strategy may include one or more instructions for suggesting the avoidance of a medication, a medical treatment, location, toxin, or environmental pollutant.

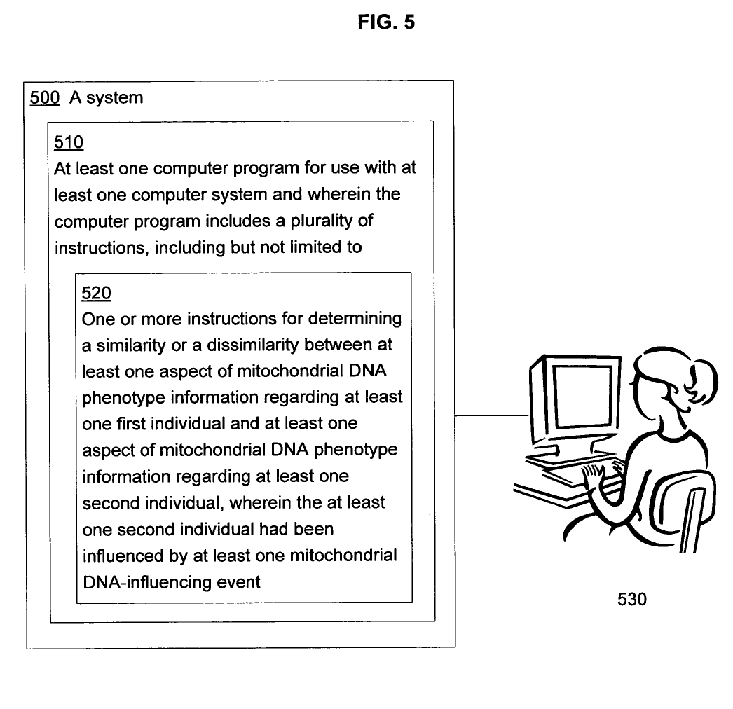

FIG. 5 depicts a partial view of a system that may serve as an illustrative environment of or for subject matter technologies. One or more users 530 may use a system 500 including at least one computer program 510 for use with at least one computer system, wherein the at least one computer program 510 includes a plurality of instructions. One or more users 530 may include, for example, one or more administrators, medical personnel, pharmacists, geneticists, researchers or technicians. Although a single user is shown in FIG. 5, in some embodiments at least one group of users or at least one series of users may interact with the system. In some embodiments, the one or more users 530 may include a computer system, artificial intelligence system (AI) or other circuitry. The at least one computer program 510 may include one or more instructions for determining a similarity or a dissimilarity between at least one aspect of mitochondrial DNA phenotype information regarding at least one first individual and at least one aspect of mitochondrial DNA phenotype information regarding at least one second individual, wherein the at least one second individual had been influenced by at least one mitochondrial DNA-influencing event 520.

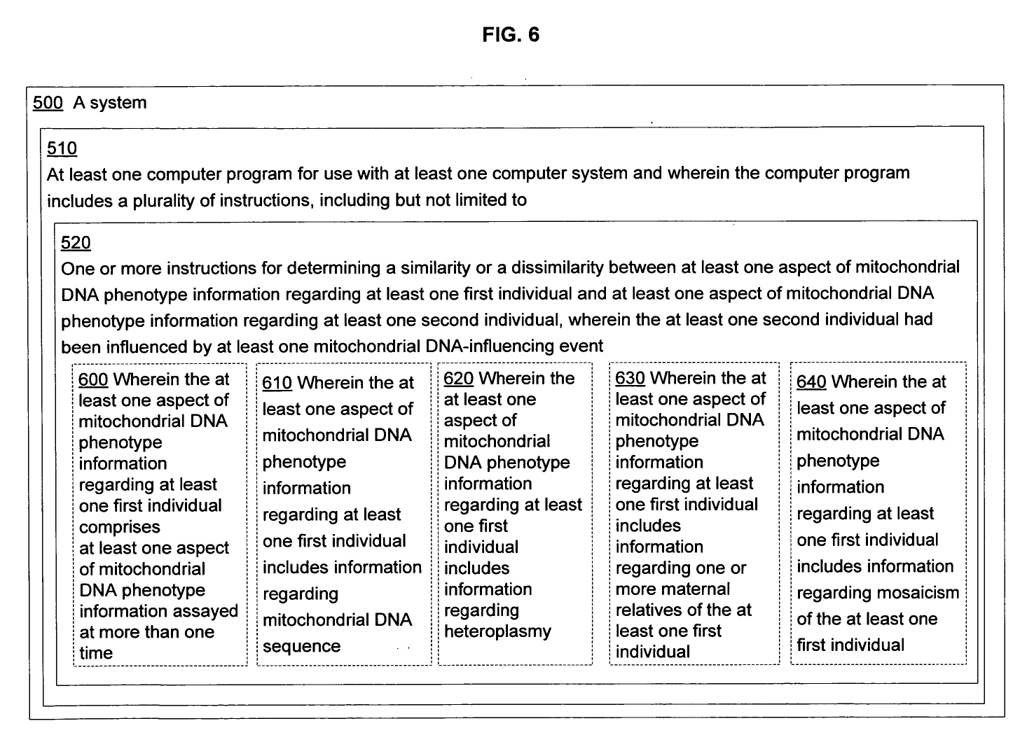

FIG. 6 illustrates further aspects of the system shown in FIG. 5. In some embodiments, the one or more instructions for determining a similarity or a dissimilarity 520 may include one or more instructions wherein the at least one aspect of mitochondrial DNA phenotype information regarding at least one first individual comprises at least one aspect of mitochondrial DNA phenotype information assayed at more than one time 600. For example, aspects of mitochondrial DNA phenotype information may be assayed on multiple occasions or set intervals, such as weekly or monthly. For example, aspects of mitochondrial DNA phenotype information may be assayed in multiple assays carried out in parallel or in series, and the information from the assays combined into a system. For example, at least one aspect of mitochondrial DNA phenotype information may be assayed at irregular intervals, such as when an individual enters into a medical treatment or consults medical personnel. In some embodiments, the one or more instructions for determining a similarity or a dissimilarity 520 may include one or more instructions wherein the at least one aspect of mitochondrial DNA phenotype information regarding at least one first individual includes information regarding mitochondrial DNA sequence 610. For example, information regarding mitochondrial DNA sequence may include information regarding linear DNA sequence such as polymorphisms or mutations. Information regarding mitochondrial DNA sequence may include, for example, information regarding the class, group or homology of one or more mitochondrial DNA sequence regions with at least one known mitochondrial DNA class(es), group(s), or sequence(s). In some embodiments, the one or more instructions for determining a similarity or a dissimilarity 520 may include one or more instructions wherein the at least one aspect of mitochondrial DNA phenotype information regarding at least one first individual includes information regarding heteroplasmy 620. Information regarding heteroplasmy, for example, may include information regarding heteroplasmy between tissue types or within tissue types, and may include information regarding heteroplasmy of an individual or multiple individuals. In some embodiments, the one or more instructions for determining a similarity or a dissimilarity 520 may include one or more instructions wherein the at least one aspect of mitochondrial DNA phenotype information regarding at least one first individual includes information regarding one or more maternal relatives of the at least one first individual 630. For example, information regarding one or more maternal relatives of the at least one individual may include information regarding the individual's biological mother, maternal grandmother, maternal aunts and uncles, or maternal cousins. In some embodiments, the one or more instructions for determining a similarity or a dissimilarity 520 may include one or more instructions wherein the at least one aspect of mitochondrial DNA phenotype information regarding at least one first individual includes information regarding mosaicism of the at least one first individual 640. For example, information regarding mosaicism of at least one first individual may include information regarding mosaicism of two or more tissue types, or mosaicism within at least one tissue type. For example, information regarding mosaicism of at least one first individual may include information such as the presence or absence of mosaicism, the location of mosaicism, the tissue or tissues involved in the mosaicism, or the proportion of various subtypes of cells in mosaic tissue.

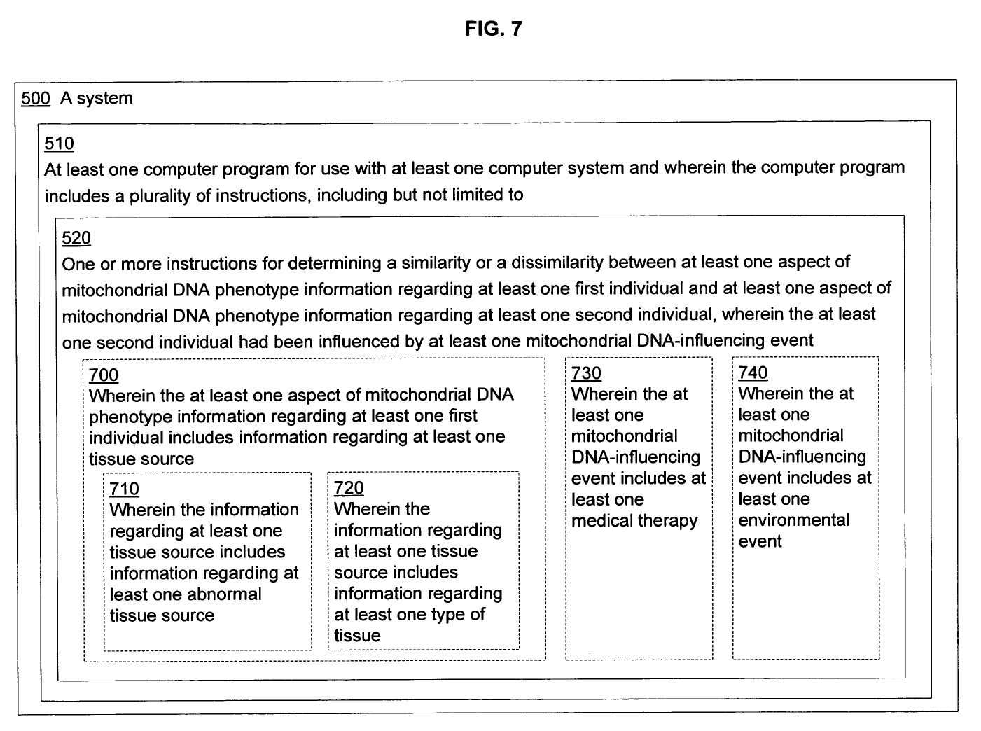

FIG. 7 depicts further aspects of the system shown in FIG. 5. In some embodiments, the one or more instructions for determining a similarity or a dissimilarity 520 may include one or more instructions wherein the at least one aspect of mitochondrial DNA phenotype information regarding at least one first individual includes information regarding at least one tissue source 700. For example, the information regarding at least one tissue source may include information regarding the origin, storage, pathology, pathological subtype, or handling of the tissue. For example, the information regarding at least one tissue source may include information regarding at least one physical, spatial or relative anatomic source. One or more instructions 700 may include wherein the information regarding at least one tissue source includes information regarding at least one abnormal tissue source 710. For example, the information regarding at least one abnormal tissue source may include information regarding a neoplastic source, a displastic source, a diseased source, an infectious source or a cancerous source. One or more instructions 700 may include wherein the information regarding at least one tissue source includes information regarding at least one type of tissue 720. For example, information regarding at least one type of tissue may include information regarding at least one clinical diagnosis, at least one pathology report, or at least one surgical report. For example, the information regarding at least one type of tissue may include the origin tissue type, the handling of the tissue, or one or more treatments to the tissue. In some embodiments, the information regarding at least one type of tissue may include cellular developmental stage, lineage, or status. For example, the information regarding at least one type of tissue may include information regarding at least one physical, spatial or relative anatomic source. See, e.g., Kujoth et al., The role of mitochondrial DNA mutations in mammalian aging, PLOS Genetics, 3: e24 (2007), which is herein incorporated by reference. In some embodiments, the one or more instructions for determining a similarity or a dissimilarity 520 may include one or more instructions wherein the at least one mitochondrial DNA-influencing event includes at least one medical therapy 730. For example, a mitochondrial DNA-influencing event may include a medical therapy including one or more medications, or one or more treatments. A mitochondrial DNA-influencing event may include at least one medical therapy including one or more energy-based procedures, for example radiation therapy, UV therapy, thermal therapy, phototherapy, or ultrasound therapy. For example, a mitochondrial DNA-influencing event may include at least one medical therapy with at least one therapeutic agent, wherein the at least one therapeutic agent may for example, include at least one drug, biologic, biological material, formulation, pharmaceutical, nutraceutical, dietary supplement, vitamin or compound. A therapeutic agent may be administered, for example, in a variety of ways including oral, topical, iv, ip, sc, intranasal, and inhalation. In some embodiments, the one or more instructions for determining a similarity or a dissimilarity 520 may include one or more instructions wherein the at least one mitochondrial DNA-influencing event includes at least one environmental event 740. For example, an environmental event may include exposure of an individual to radiation, one or more pollutants, or one or more toxins. For example, an environmental event may include exposure of an individual to excessive levels of cigarette smoke. For example, an environmental event may include exposure of an individual to excessive levels of toxins such as asbestos, or arsenic.

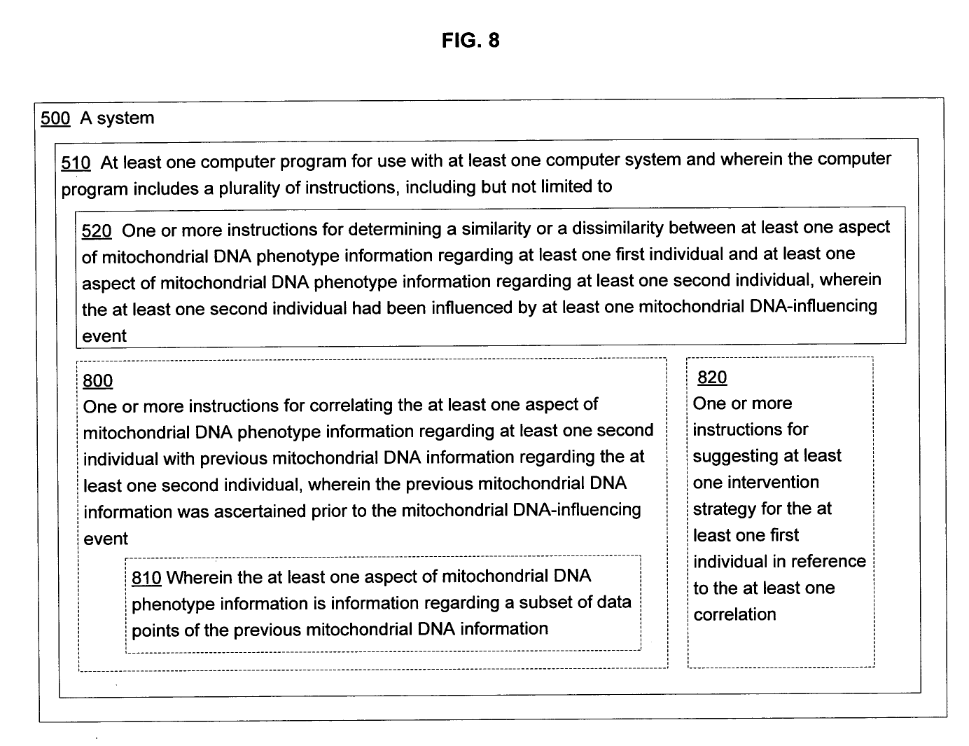

FIG. 8 depicts further aspects of the system shown in FIG. 5. In some embodiments, the computer program 520 may include one or more instructions for correlating the at least one aspect of mitochondrial DNA phenotype information regarding at least one second individual with previous mitochondrial DNA information regarding the at least one second individual, wherein the previous mitochondrial DNA information was ascertained prior to the mitochondrial DNA-influencing event 800. In some embodiments, the one or more instructions 800 may include wherein the at least one aspect of mitochondrial DNA phenotype information is information regarding a subset of data points of the previous mitochondrial DNA information 810. For example, an initial assay or group of assays may yield information regarding a large number of data points, while subsequent smaller assays or groups of assays may yield information regarding a smaller group, or subset of data points. For example, an initial assay may yield a large number of data points that are included in the system, while information from further assays that is incorporated into a system is limited to a subset of data points. The computer program 510 may also include one or more instructions for suggesting at least one intervention strategy for the at least one first individual in reference to the at least one correlation 820. For example, one or more instructions for suggesting at least one intervention strategy may include one or more instructions for suggesting a course of medication, or a medical treatment. For example, one or more instructions for suggesting at least one intervention strategy may include one or more instructions for suggesting the avoidance of a medication, a medical treatment, location, toxin, or environmental pollutant.

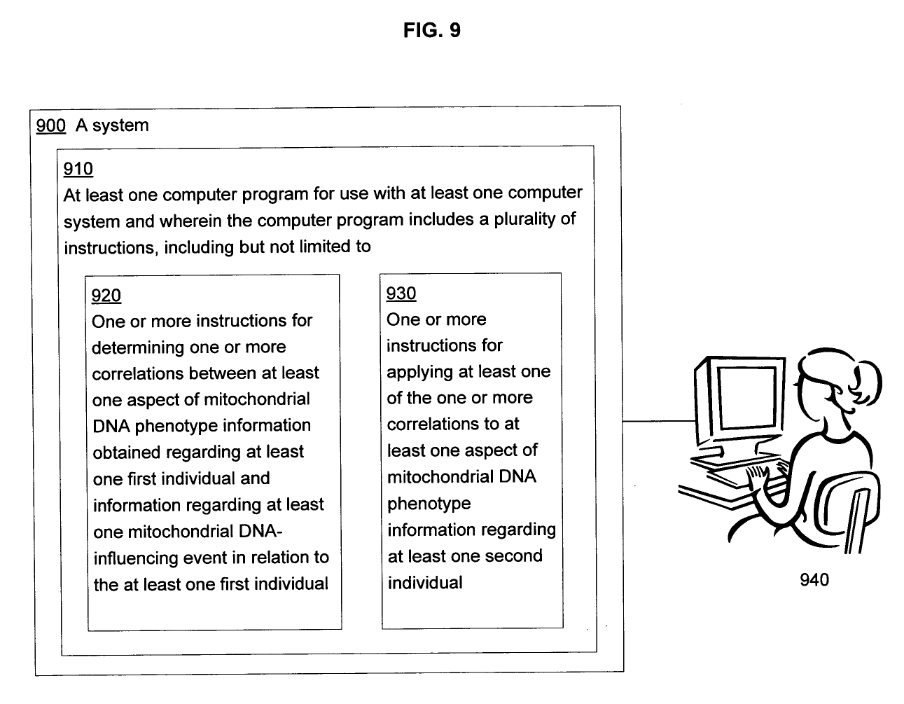

FIG. 9 depicts a partial view of a system that may serve as an illustrative environment of or for subject matter technologies. One or more users 940 may use a system 900 including at least one computer program 910 for use with at least one computer system, wherein the at least one computer program 910 includes a plurality of instructions. One or more users 940 may include, for example, one or more administrators, medical personnel, pharmacists, geneticists, researchers or technicians. Although a single user is shown in FIG. 9, in some embodiments at least one group of users or at least one series of users may interact with the system. In some embodiments, the one or more users 940 may include a computer system, artificial intelligence system (AI) or other circuitry. The at least one computer program 910 may include one or more instructions for determining one or more correlations between at least one aspect of mitochondrial DNA phenotype information obtained regarding at least one first individual and information regarding at least one mitochondrial DNA-influencing event in relation to the at least one first individual 920. The at least one computer program 910 may include one or more instructions for applying at least one of the one or more correlation to at least one aspect of mitochondrial DNA phenotype information regarding at least one second individual 930.

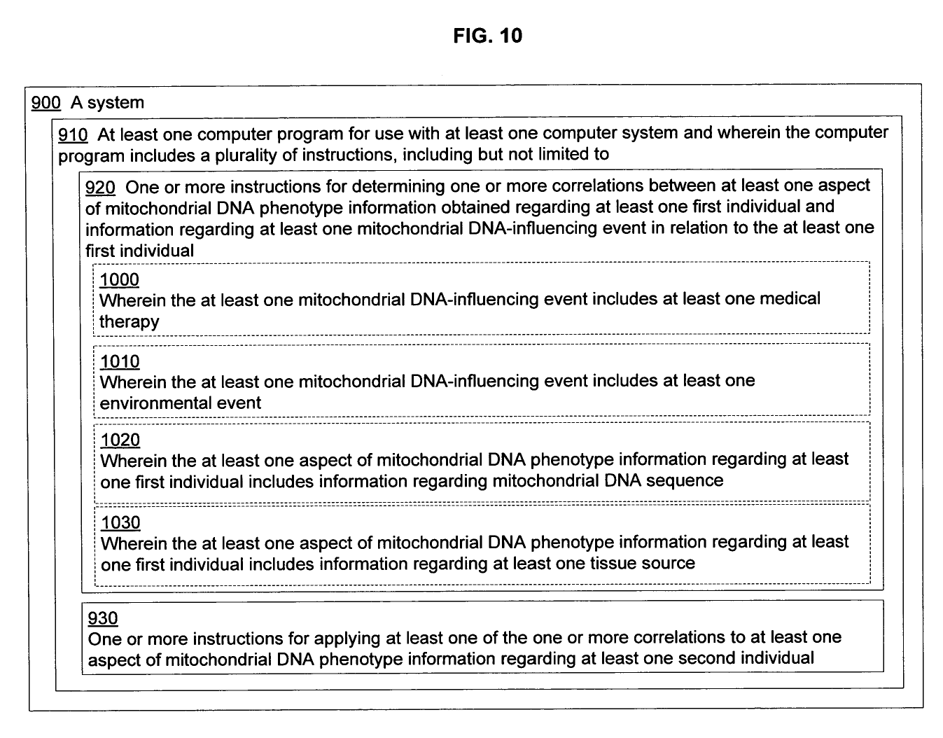

FIG. 10 depicts further aspects of the system shown in FIG. 9. In some embodiments, one or more instructions for determining one or more correlations 920 may include wherein the at least one mitochondrial DNA-influencing event includes at least one medical therapy 1000. For example, a mitochondrial DNA-influencing event may include a medical therapy including one or more medications, or one or more treatments. A mitochondrial DNA-influencing event may include at least one medical therapy including one or more energy-based procedures, for example radiation therapy, UV therapy, thermal therapy, phototherapy, or ultrasound therapy. For example, a mitochondrial DNA-influencing event may include at least one medical therapy with at least one therapeutic agent, wherein the at least one therapeutic agent may for example, include at least one drug, biologic, biological material, formulation, pharmaceutical, nutraceutical, dietary supplement, vitamin or compound. A therapeutic agent may be administered, for example, in a variety of ways including oral, topical, iv, ip, sc, intranasal, and inhalation. In some embodiments, one or more instructions for determining one or more correlations 920 may include wherein the at least one mitochondrial DNA-influencing event includes at least one environmental event 1010. For example, an environmental event may include exposure of an individual to radiation, one or more pollutants, or one or more toxins. For example, an environmental event may include exposure of an individual to excessive levels of cigarette smoke. For example, an environmental event may include exposure of an individual to excessive levels of toxins such as asbestos, or arsenic. In some embodiments, an environmental event may include some element of activity by an individual, such as when an individual goes to or stays in a location with exposures such as those described herein. In some embodiments, one or more instructions for determining one or more correlations 920 may include wherein the at least one aspect of mitochondrial DNA phenotype information regarding at least one first individual includes information regarding mitochondrial DNA sequence 1020. For example, information regarding mitochondrial DNA sequence may include information regarding linear DNA sequence such as polymorphisms or mutations. Information regarding mitochondrial DNA sequence may include, for example, information regarding the class, group or homology of one or more mitochondrial DNA sequence regions with at least one known mitochondrial DNA class(es), group(s), or sequence(s). In some embodiments, one or more instructions for determining one or more correlations 920 may include wherein the at least one aspect of mitochondrial DNA phenotype information regarding at least one first individual includes information regarding at least one tissue source 1030. For example, the information regarding at least one tissue source may include information regarding the origin, storage, pathology, pathological subtype, or handling of the tissue. For example, the information regarding at least one tissue source may include information regarding at least one physical, spatial or relative anatomic source.

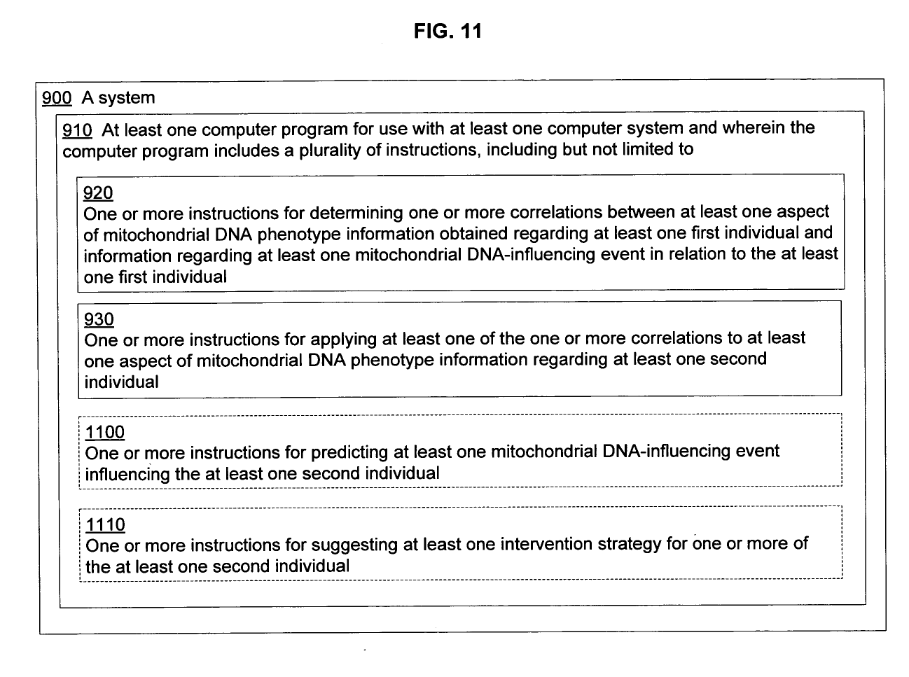

FIG. 11 shows aspects of the system depicted in FIG. 10. In some embodiments, the at least one computer program 910 may include one or more instructions for predicting at least one mitochondrial DNA-influencing event influencing the at least one second individual 1100. In some embodiments, the at least one computer program 910 may include one or more instructions for suggesting at least one intervention strategy for one or more of the at least one second individual 1110. For example, one or more instructions for suggesting at least one intervention strategy may include instructions for suggesting at least one medication, therapy, treatment, or course of action relating to the at least one second individual.

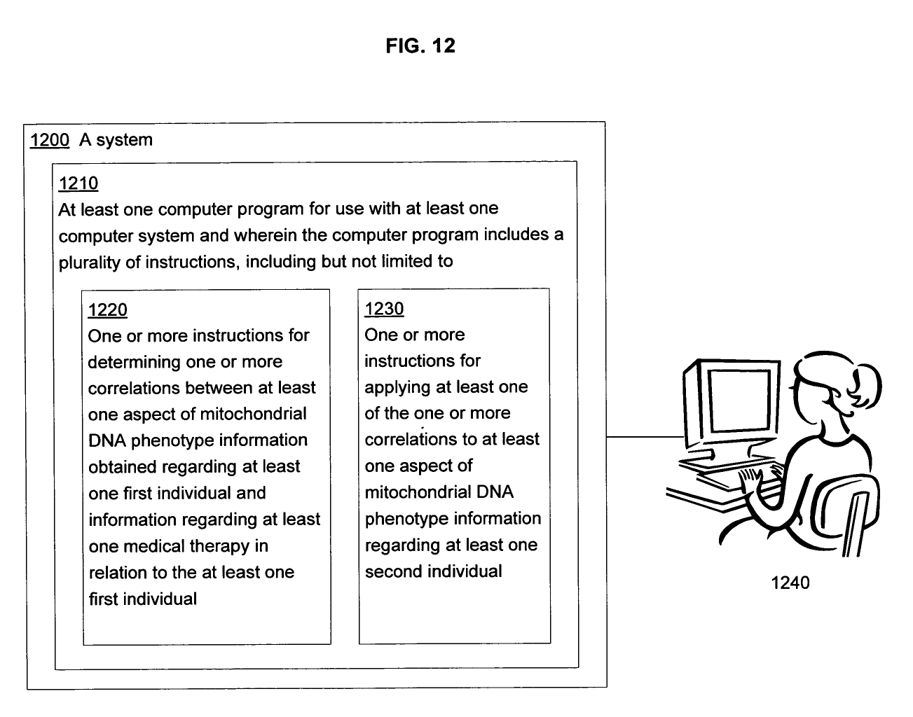

FIG. 12 depicts a partial view of a system that may serve as an illustrative environment of or for subject matter technologies. One or more users 1240 may use a system 1200 including at least one computer program 1210 for use with at least one computer system, wherein the at least one computer program 1210 includes a plurality of instructions. One or more users 1240 may include, for example, one or more administrators, medical personnel, pharmacists, geneticists, researchers or technicians. Although a single user is shown in FIG. 12, in some embodiments at least one group of users or at least one series of users may interact with the system. In some embodiments, the one or more users 1240 may include a computer system, artificial intelligence system (AI) or other circuitry. The at least one computer program 1210 may include one or more instructions for determining one or more correlations between at least one aspect of mitochondrial DNA phenotype information obtained regarding at least one first individual and information regarding at least one medical therapy in relation to the at least one first individual 1220. For example, a medical therapy may include one or more medications, or one or more treatments. At least one medical therapy may include one or more energy-based procedures, for example radiation therapy, UV therapy, thermal therapy, phototherapy, or ultrasound therapy. The at least one computer program 1210 may include one or more instructions for applying at least one of the one or more correlations to at least one aspect of mitochondrial DNA phenotype information regarding at least one second individual 1230.

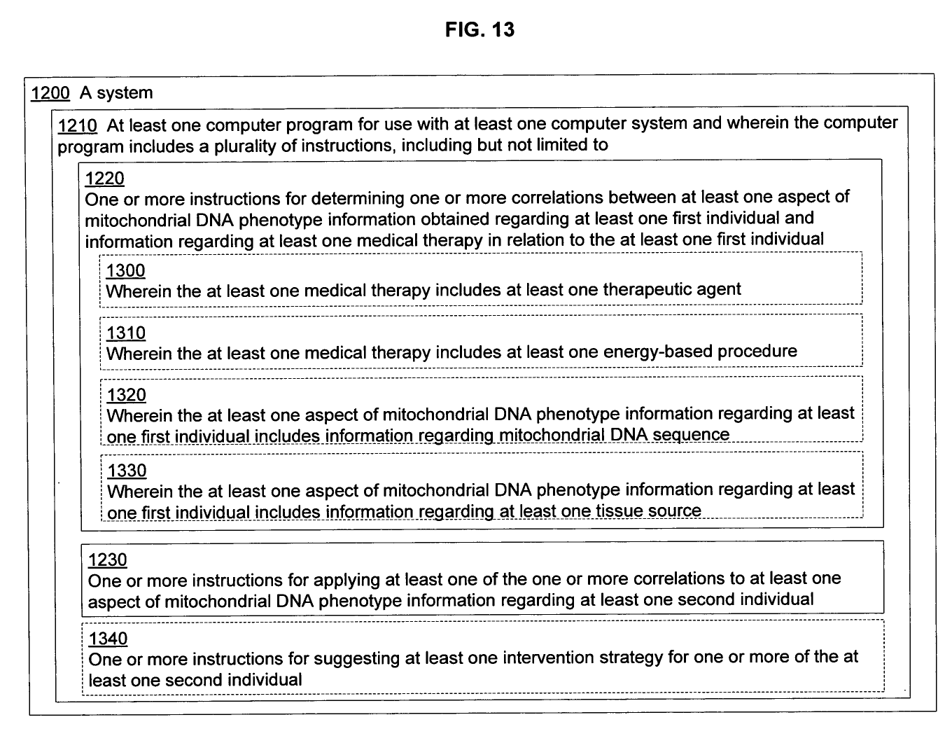

FIG. 13 shows further aspects of the system shown in FIG. 12. In some embodiments, the one or more instructions for determining one or more correlations 1220 may include wherein the at least one medical therapy includes at least one therapeutic agent 1300. In some embodiments, the one or more instructions for determining one or more correlations 1220 may include wherein the at least one medical therapy includes at least one energy-based procedure 1310. For example, the at least one medical therapy may include at least one energy-based procedure such as radiation therapy, ultrasound therapy, sonic therapy, UV therapy, phototherapy, or thermal therapy. In some embodiments, the one or more instructions for determining one or more correlations 1220 may include wherein the at least one aspect of mitochondrial DNA phenotype information regarding at least one first individual includes information regarding mitochondrial DNA sequence 1320. For example, information regarding mitochondrial DNA sequence may include information regarding linear DNA sequence such as polymorphisms or mutations. Information regarding mitochondrial DNA sequence may include, for example, information regarding the class, group or homology of one or more mitochondrial DNA sequence regions with at least one known mitochondrial DNA class(es), group(s), or sequence(s). In some embodiments, the one or more instructions for determining one or more correlations 1220 may include wherein the at least one aspect of mitochondrial DNA phenotype information regarding at least one first individual includes information regarding at least one tissue source 1330. For example, the information regarding at least one tissue source may include information regarding the origin, storage, pathology, pathological subtype, or handling of the tissue. For example, the information regarding at least one tissue source may include information regarding at least one physical, spatial or relative anatomic source. In some embodiments, the at least one computer program 1210 may include one or more instructions for suggesting at least one intervention strategy for one or more of the at least one second individual 1340. For example, one or more instructions for suggesting at least one intervention strategy may include instructions for suggesting at least one medication, therapy, treatment, or course of action relating to the at least one second individual.

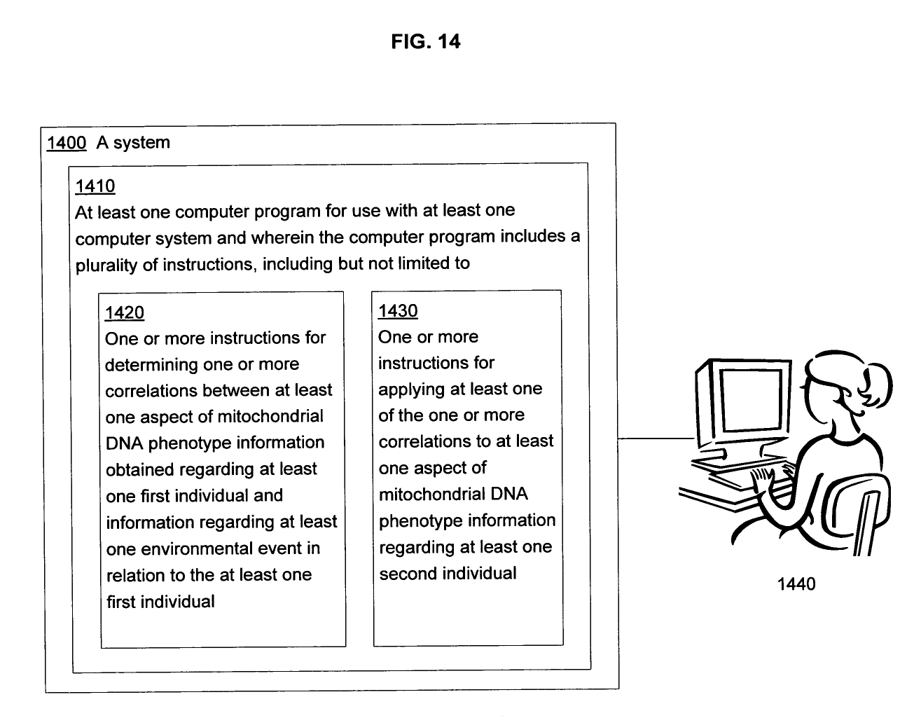

FIG. 14 depicts a partial view of a system that may serve as an illustrative environment of or for subject matter technologies. One or more users 1440 may use a system 1400 including at least one computer program 1410 for use with at least one computer system, wherein the at least one computer program 1410 includes a plurality of instructions. One or more users 1440 may include, for example, one or more administrators, medical personnel, pharmacists, geneticists, researchers or technicians. Although a single user is shown in FIG. 14, in some embodiments at least one group of users or at least one series of users may interact with the system. In some embodiments, the one or more users 1440 may include a computer system, artificial intelligence system (AI) or other circuitry. The at least one computer program 1410 may include one or more instructions for determining one or more correlations between at least one aspect of mitochondrial DNA phenotype information obtained regarding at least one first individual and information regarding at least one environmental event in relation to the at least one first individual 1420. For example, an environmental event may include exposure of an individual to radiation, one or more pollutants, or one or more toxins. In some embodiments, an environmental event may include some element of activity by an individual, such as when an individual goes to or stays in a location with exposures such as those described herein. The at least one computer program 1410 may include one or more instructions for applying at least one of the one or more correlations to at least one aspect of mitochondrial DNA phenotype information regarding at least one second individual 1430.

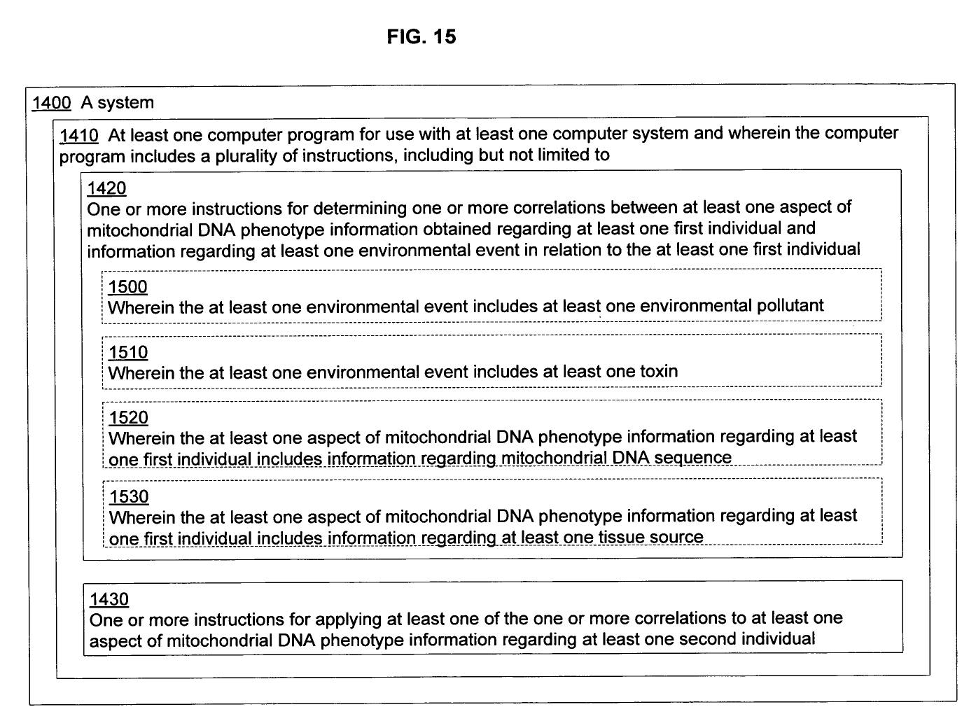

FIG. 15 illustrates further aspects of the system shown in FIG. 14. In some embodiments, the one or instructions for determining one or more correlations 1420 may include wherein the at least environmental event includes at least one environmental pollutant 1500. For example, the at least one environmental event may include an environmental pollutant such as overly high levels of arsenic in drinking water. For example, the at least one environmental event may include an environmental pollutant such as cigarette smoke or asbestos particles in the air. In some embodiments, an environmental event may include some element of activity by an individual, such as when an individual goes to or stays in a location with exposures such as those described herein. In some embodiments, the one or instructions for determining one or more correlations 1420 may include wherein the at least one environmental event includes at least one toxin 1510. For example, the at least one environmental event may include at least one toxin such as released industrial wastes, unprocessed sewage, contaminants such as arsenic, or particulates in the air, including asbestos. In some embodiments, the one or instructions for determining one or more correlations 1420 may include wherein the at least one aspect of mitochondrial DNA phenotype information regarding at least one first individual includes information regarding mitochondrial DNA sequence 1520. For example, information regarding mitochondrial DNA sequence may include information regarding linear DNA sequence such as polymorphisms or mutations. Information regarding mitochondrial DNA sequence may include, for example, information regarding the class, group or homology of one or more mitochondrial DNA sequence regions with at least one known mitochondrial DNA class(es), group(s), or sequence(s). In some embodiments, the one or instructions for determining one or more correlations 1420 may include wherein the at least one aspect of mitochondrial DNA phenotype information regarding at least one first individual includes information regarding at least one tissue source 1530. For example, the information regarding at least one tissue source may include information regarding the origin, storage, pathology, pathological subtype, or handling of the tissue. For example, the information regarding at least one tissue source may include information regarding at least one physical, spatial or relative anatomic source.

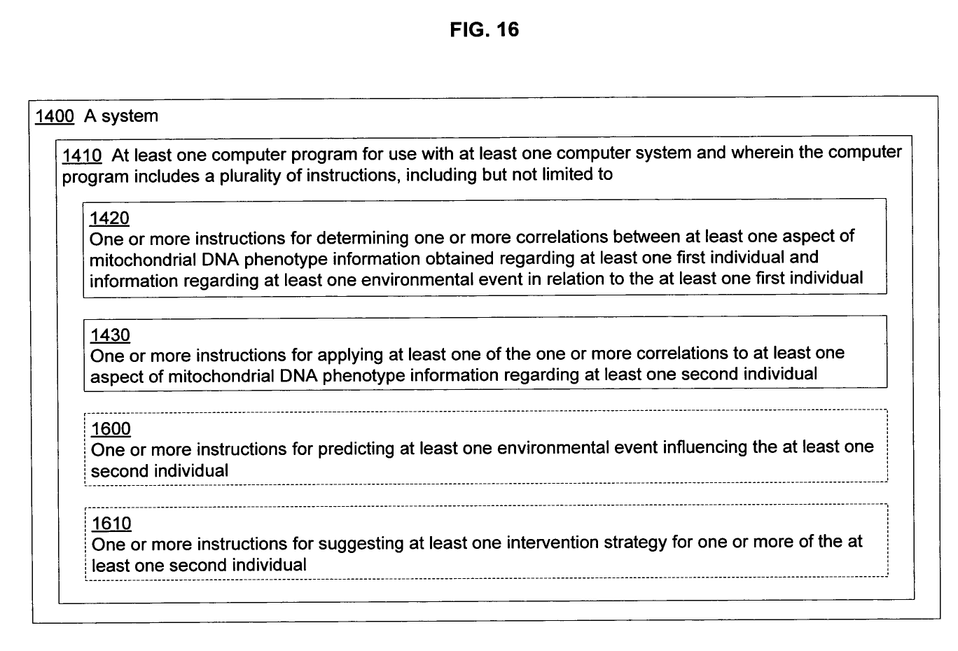

FIG. 16 depicts further aspects of the system illustrated in FIG. 14. In some embodiments, at least one computer program 1410 may include one or more instructions for predicting at least one environmental event influencing the at least one second individual 1600. For example, one or more instructions for predicting at least one environmental event influencing the at least one second individual may include instructions for predicting the influence of at least one toxin or pollutant. In some embodiments, at least one computer program 1410 may include one or more instructions for suggesting at least one intervention strategy for one or more of the at least one second individual 1610. For example, one or more instructions for suggesting at least one intervention strategy may include instructions for suggesting at least one medication, therapy, treatment or course of action relating to the at least one second individual.

### B. Operation(s) or Process(es)

Following are a series of flowcharts depicting implementations of processes. For ease of understanding, the flowcharts are organized such that the initial flowcharts present implementations via an overall “big picture” or “top-level” viewpoint and thereafter the subsequent flowcharts present alternate implementations or expansions of the “big picture” flowcharts as either sub-steps or additional steps building on one or more earlier-presented flowcharts. Those having skill in the art will appreciate that the style of presentation utilized herein (e.g., beginning with a presentation of a flowchart(s) presenting an overall view and thereafter providing additions to or further details in subsequent flowcharts) generally allows for a more rapid and reliable understanding of the various process implementations.

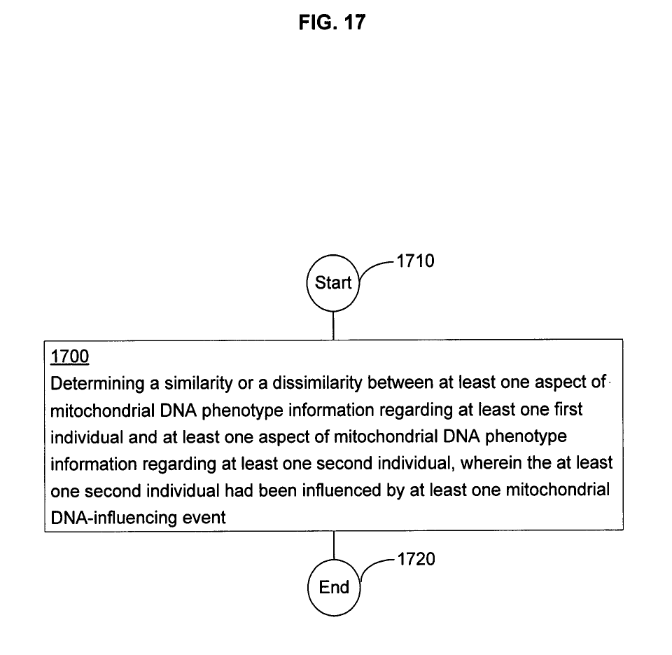

With reference now to FIG. 17, illustrated is a flowchart of a method. Block 710 depicts the start of the method. Block 1700 depicts determining a similarity or dissimilarity between at least one aspect of mitochondrial DNA phenotype information regarding at least one first individual and at least one aspect of mitochondrial DNA phenotype information regarding at least one second individual, wherein the at least one second individual had been influenced by the at least one mitochondrial DNA-influencing event. Block 1720 depicts the end of the method.

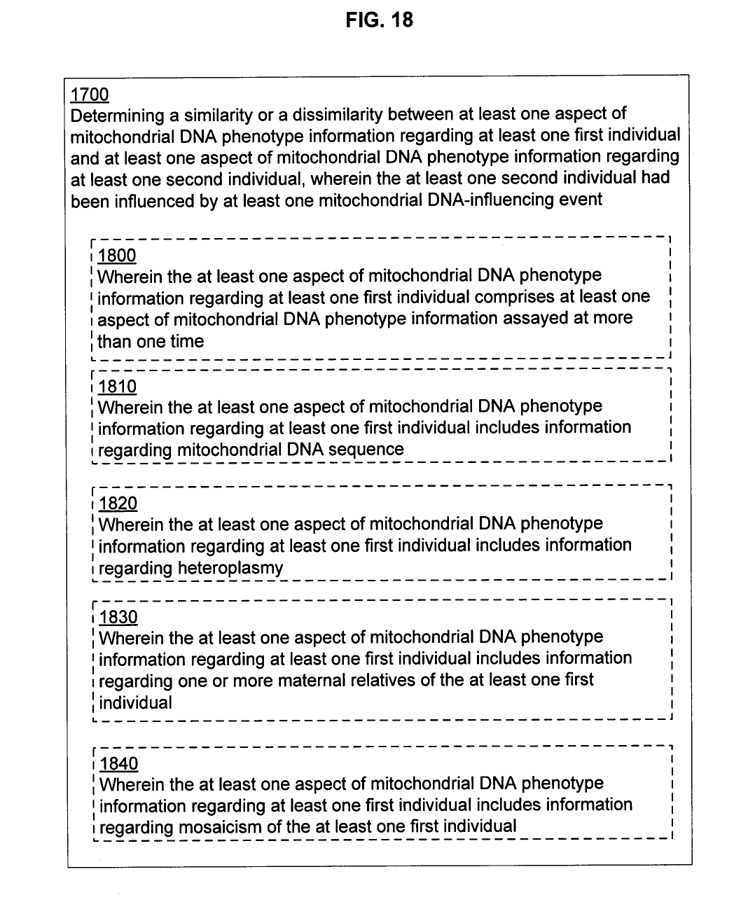

FIG. 18 depicts alternate aspects of the flowchart illustrated in FIG. 17. In some embodiments, block 1700 may include one or more of optional blocks 1800, 1810, 1820, 1830 or 1840. Block 1800 depicts wherein the at least one aspect of mitochondrial DNA phenotype information regarding at least one first individual comprises at least one aspect of mitochondrial DNA phenotype information assayed at more than one time. Block 1810 illustrates wherein the at least one aspect of mitochondrial DNA phenotype information regarding at least one first individual includes information regarding mitochondrial DNA sequence. Block 1820 shows wherein the at least one aspect of mitochondrial DNA phenotype information regarding at least one first individual includes information regarding heteroplasmy. Block 1830 depicts wherein the at least one aspect of mitochondrial DNA phenotype information regarding at least one first individual includes information regarding one or more maternal relatives of the at least one first individual. Block 1840 illustrates wherein the at least one aspect of mitochondrial DNA phenotype information regarding at least one first individual includes information regarding mosaicism of the at least one first individual.

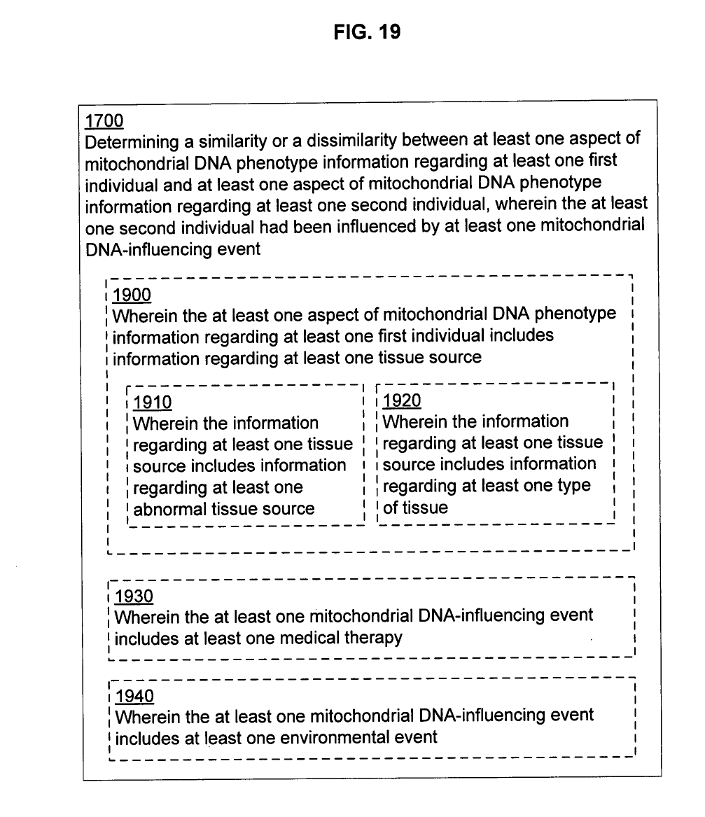

FIG. 19 illustrates alternate aspects of the flowchart illustrated in FIG. 17. In some embodiments, block 1700 may include one or more of optional blocks 1900, 1910, 1920, 1930 or 1940. Block 1900 depicts wherein the at least one aspect of mitochondrial DNA phenotype information regarding at least one first individual includes information regarding at least one tissue source. Block 1900 may include block 1910 illustrating wherein the information regarding at least one tissue source includes information regarding at least one abnormal tissue source. Block 1900 may include block 1910 showing wherein the information regarding at least one tissue source includes information regarding at least one type of tissue. Block 1700 may also include block 1930 depicting wherein the at least one mitochondrial DNA-influencing event includes at least one medical therapy. Block 1700 may include block 1940 illustrating wherein the at least one mitochondrial DNA-influencing event includes at least one environmental event.

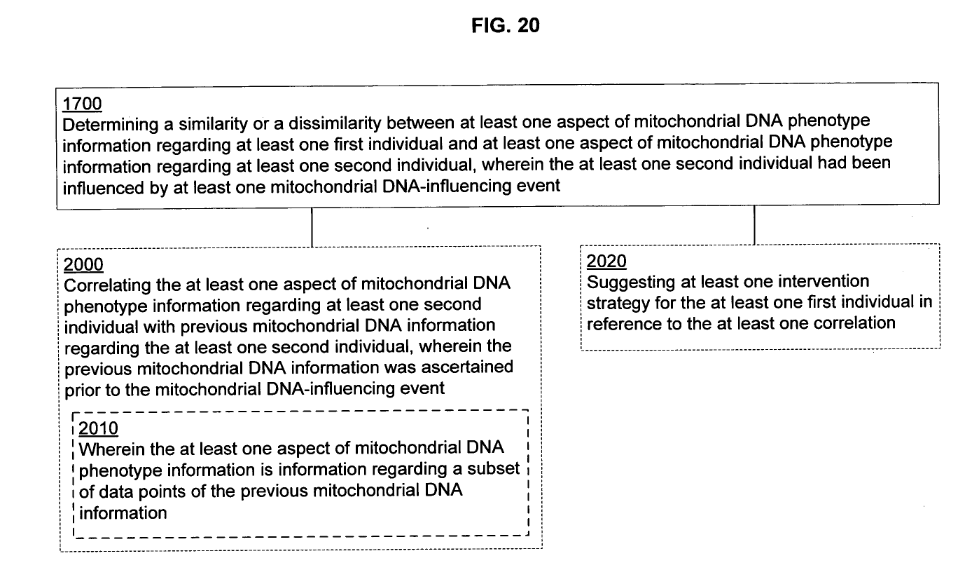

FIG. 20 depicts alternate aspects of the flowchart illustrated in FIG. 17. As shown in FIG. 20, the flowchart may include one or more of blocks 2000, 2010, or 2020. Block 2000 illustrates correlating the at least one aspect of mitochondrial DNA phenotype information regarding at least one second individual with previous mitochondrial DNA information regarding the at least one second individual, wherein the previous mitochondrial DNA information was ascertained prior to the mitochondrial DNA-influencing event. Block 2000 may include block 2010, depicting wherein the at least one aspect of mitochondrial DNA phenotype information is information regarding a subset of data points of the previous mitochondrial DNA information. The flowchart may also contain block 2020, showing suggesting at least one intervention strategy for the at least one first individual in reference to the at least one correlation.

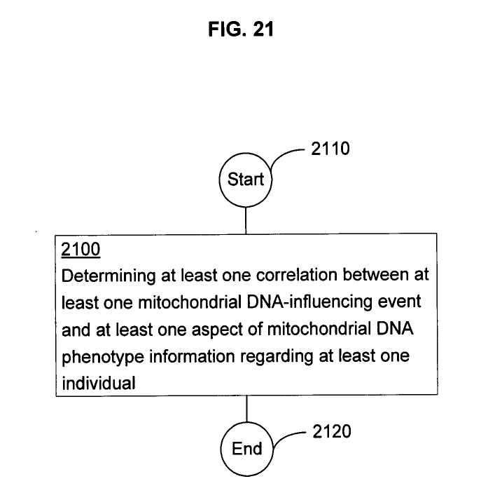

FIG. 21 illustrates a flowchart of a method. Block 2110 depicts the start of the method. Block 2100 shows determining at least one correlation between at least one mitochondrial DNA-influencing event and at least one aspect of mitochondrial DNA phenotype information regarding at least one individual. Block 2120 illustrates the end of the method.

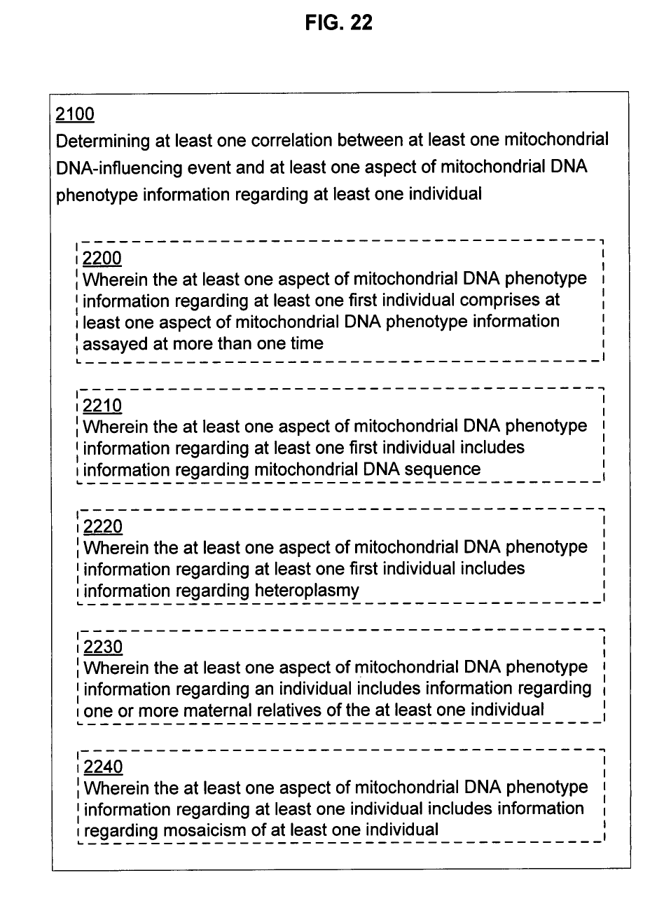

FIG. 22 depicts alternate aspects of the flowchart illustrated in FIG. 21. As shown in FIG. 22, block 2100 may include one or more of blocks 2200, 2210, 2220, 2230, or 2240. Block 2200 depicts wherein the at least one aspect of mitochondrial DNA phenotype information regarding at least one first individual comprises at least one aspect of mitochondrial DNA phenotype information assayed at more than one time. Block 2210 illustrates wherein the at least one aspect of mitochondrial DNA phenotype information regarding at least one first individual includes information regarding DNA sequence. Block 2220 depicts wherein the at least one aspect of mitochondrial DNA phenotype information regarding at least one first individual includes information regarding heteroplasmy. Block 2230 shows where in the at least one aspect of mitochondrial DNA phenotype information regarding an individual includes information regarding one or more maternal relatives of the at least one individual. Block 2240 illustrates wherein the at least one aspect of mitochondrial DNA phenotype information regarding an individual includes information regarding mosaicism of at least one individual.

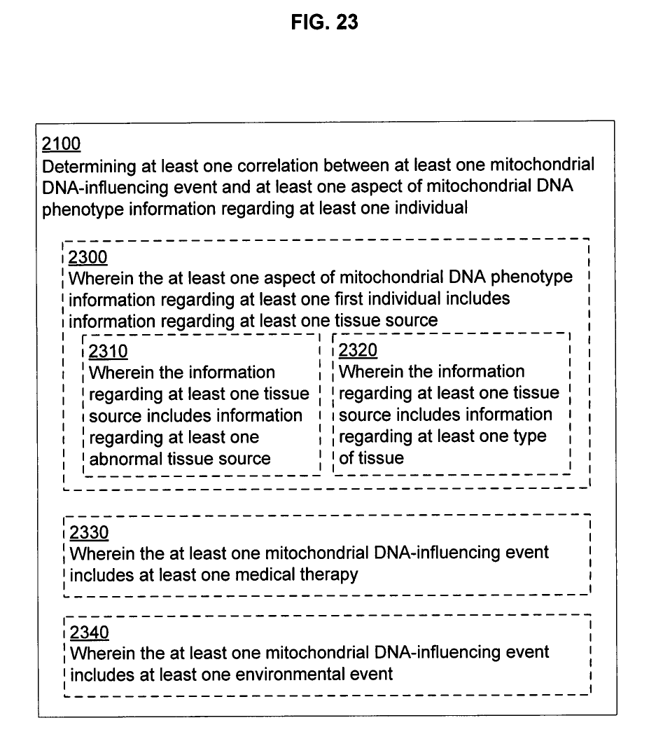

FIG. 23 illustrates alternate aspects of the flowchart shown in FIG. 21. Block 2100 may include one or more of blocks 2300, 2310, 2320, 2330, or 2340. Block 2300 depicts wherein the at least one aspect of mitochondrial DNA phenotype information regarding at least one first individual includes information regarding at least one tissue source. Block 2300 may include one or more of blocks 2310 and 2320. Block 2310 shows wherein the information regarding at least one tissue source includes information regarding at least one abnormal tissue source. Block 2320 illustrates wherein the information regarding at least one tissue source includes information regarding at least one type of tissue. Block 2100 may also include block 2330. Block 2330 depicts wherein the at least one mitochondrial DNA-influencing event includes at least one medical therapy. Block 2340 illustrates wherein the at least one mitochondrial DNA-influencing event includes at least one environmental event.

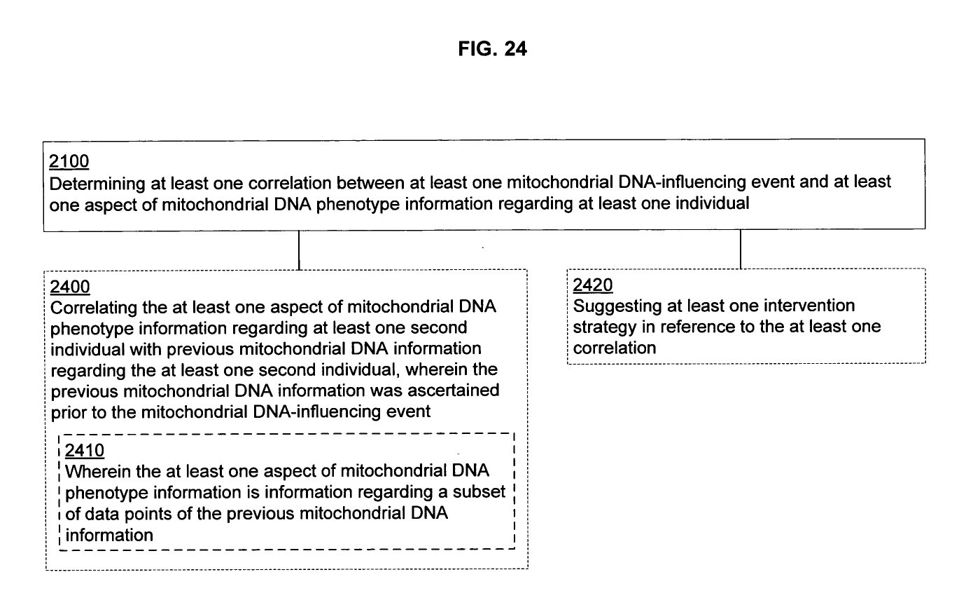

FIG. 24 depicts further aspects of the flowchart shown in FIG. 21. In addition to block 2100, a flowchart of a method may also include one or more of blocks 2400, 2410, or 2420. Block 2400 depicts correlating the at least one aspect of mitochondrial DNA phenotype information regarding at least one second individual with previous mitochondrial DNA information regarding the at least one second individual, wherein the previous mitochondrial DNA information was ascertained prior to the mitochondrial DNA-influencing event. Block 2400 may include block 2410. Block 2410 illustrates wherein the at least one aspect of mitochondrial DNA phenotype information is information regarding a subset of data points of the previous mitochondrial DNA information. A flowchart may also include block 2420. Block 2420 shows suggesting at least one intervention strategy for the at least one first individual in reference to the at least one correlation.

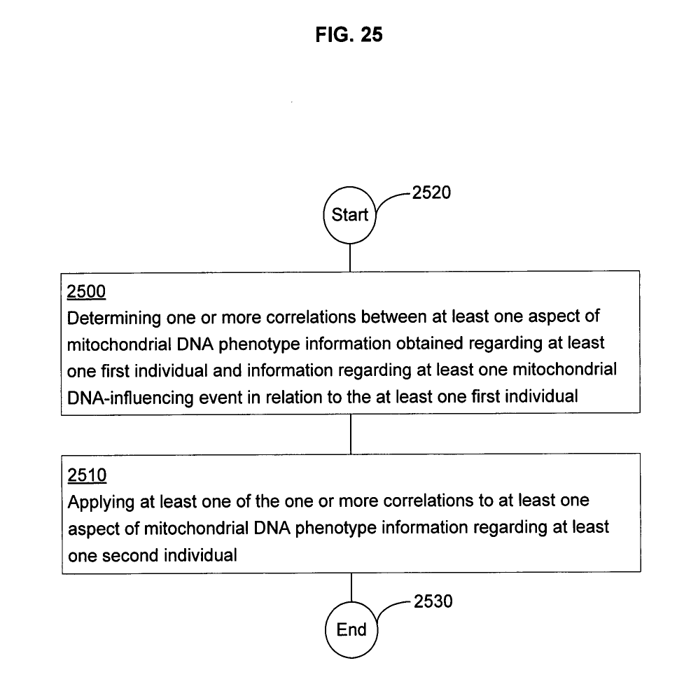

FIG. 25 illustrates a flowchart of a method. Block 2520 depicts the start of the method. Block 2500 shows determining a similarity or a dissimilarity between at least one aspect of mitochondrial DNA phenotype information obtained regarding at least one first individual and information regarding at least one mitochondrial AND-influencing event in relation to the at least one first individual. Block 2510 depicts applying at least on of the one or more correlations to at least one aspect of mitochondrial DNA phenotype information regarding at least one second individual. Block 2530 shows the end of the method.

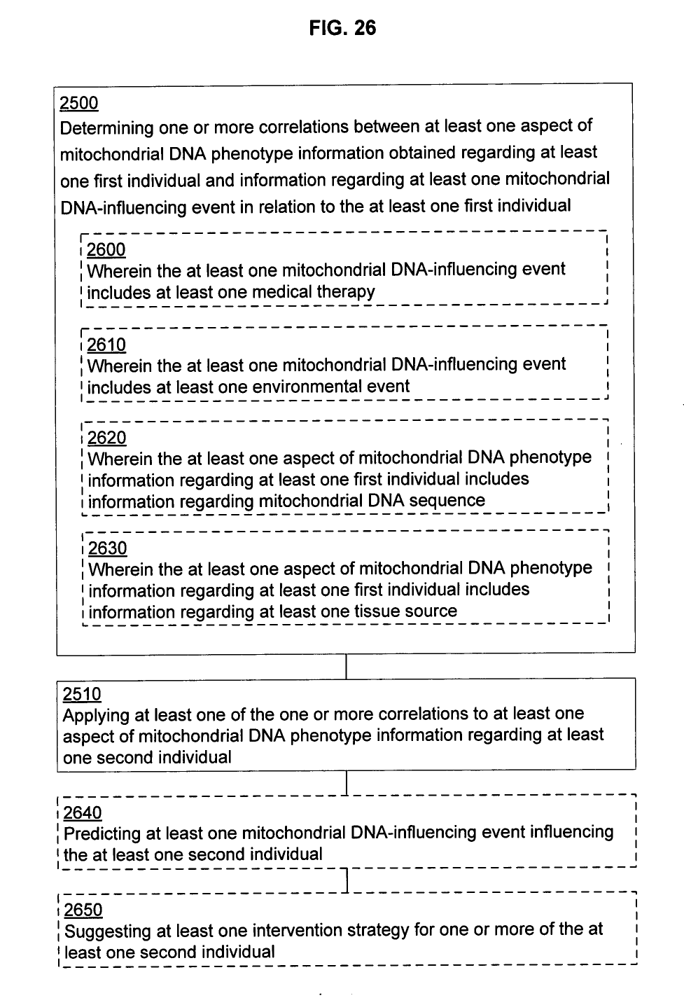

FIG. 26 illustrates further aspects of the flowchart depicted in FIG. 25. Block 2500 may include one or more of blocks 2600, 2610, 2620, or 2630. Block 2600 depicts wherein the at least one mitochondrial DNA-influencing event includes at least one medical therapy. Block 2610 shows wherein the at least one mitochondrial DNA-influencing event includes at least one environmental event. Block 2620 illustrates where the at least one aspect of mitochondrial DNA phenotype information regarding at least one first individual includes information regarding mitochondrial DNA sequence. Block 2630 depicts wherein the at least one aspect of mitochondrial DNA phenotype information regarding at least one first individual includes information regarding at least one tissue source. The flowchart may also include one or more of blocks 2640 and 2650. Block 2640 depicts predicting at least one mitochondrial DNA-influencing event influencing the at least one second individual. Block 2650 illustrates suggesting at least one intervention strategy for one or more of the at least one second individual.

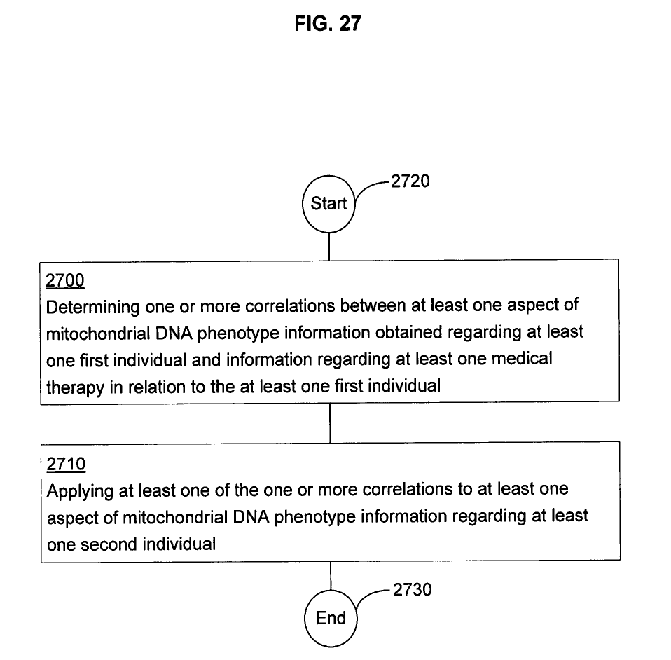

FIG. 27 depicts aspects of a method. Block 2720 shows the start of the method. Block 2700 illustrates determining one or more correlations between at least one aspect of mitochondrial DNA phenotype information obtained regarding at least one first individual and information regarding at least one medical therapy in relation to the at least one first individual. Block 2710 depicts applying at least one of the one or more correlation to at least one aspect of mitochondrial DNA phenotype information regarding at least one second individual. Block 2730 illustrates the end of the method.

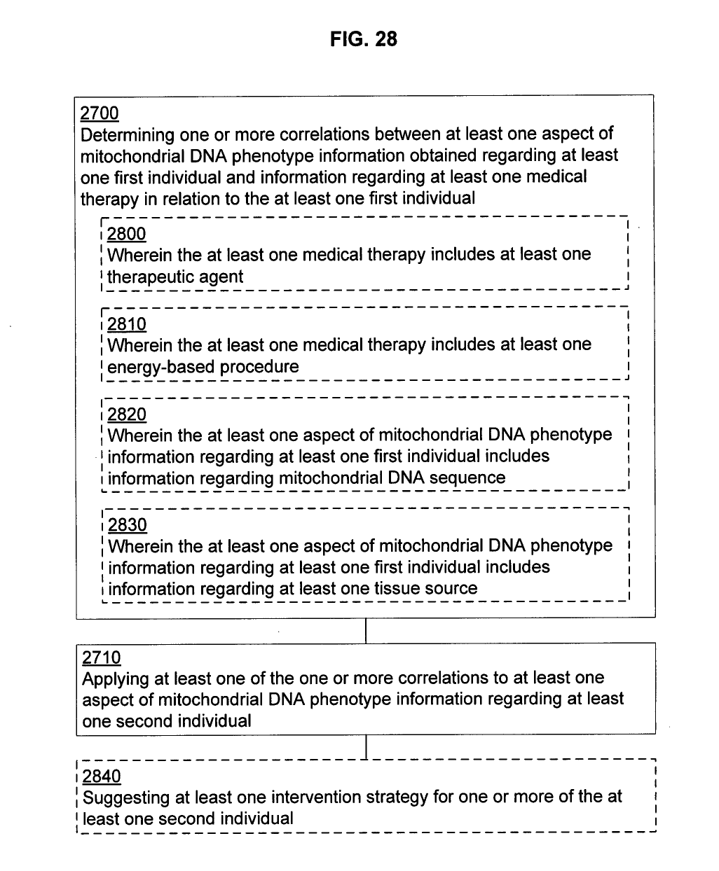

FIG. 28 illustrates further aspects of the flowchart depicted in FIG. 27. Block 2700 may include one or more of blocks 2800, 2810, 2820, or 2830. Block 2800 depicts wherein the at least one medical therapy includes at least one therapeutic agent. Block 2810 shows wherein the at least one medical therapy includes at least one energy-based procedure. Block 2820 illustrates wherein the at least one aspect of mitochondrial DNA phenotype information regarding at least one first individual includes information regarding mitochondrial DNA sequence. Block 2830 shows wherein the at least one aspect of mitochondrial DNA phenotype information regarding at least one first individual includes information regarding at least one tissue source. The flowchart may also include block 2840. Block 2840 illustrates suggesting at least one intervention strategy for one or more of the at least one second individual.

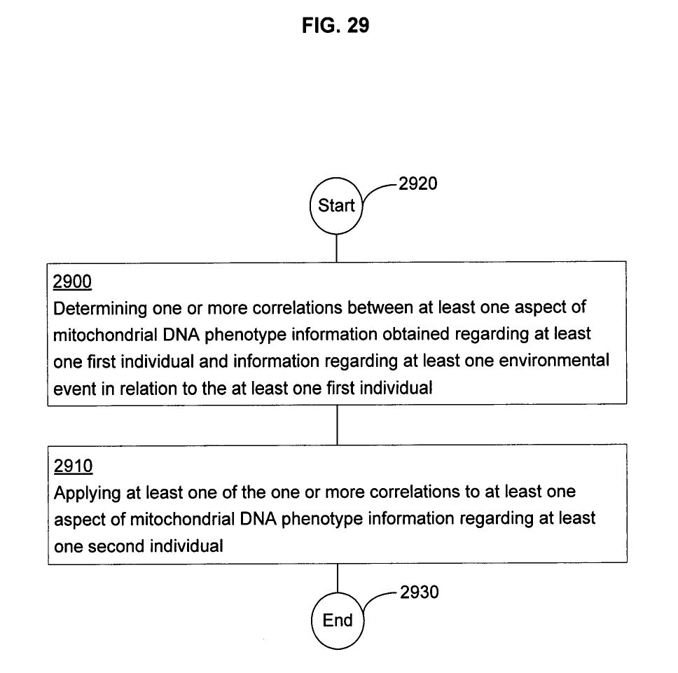

FIG. 29 shows aspects of a method. Block 2920 depicts the start of the method. Block 2900 illustrates determining one or more correlations between at least one aspect of mitochondrial DNA phenotype information obtained regarding at least one first individual and information regarding at least one environmental event in relation to the at least one first individual. Block 2910 shows applying at least one of the one or more correlations to at least one aspect of mitochondrial DNA phenotype information regarding at least one second individual. Block 2930 shows the end of the method.

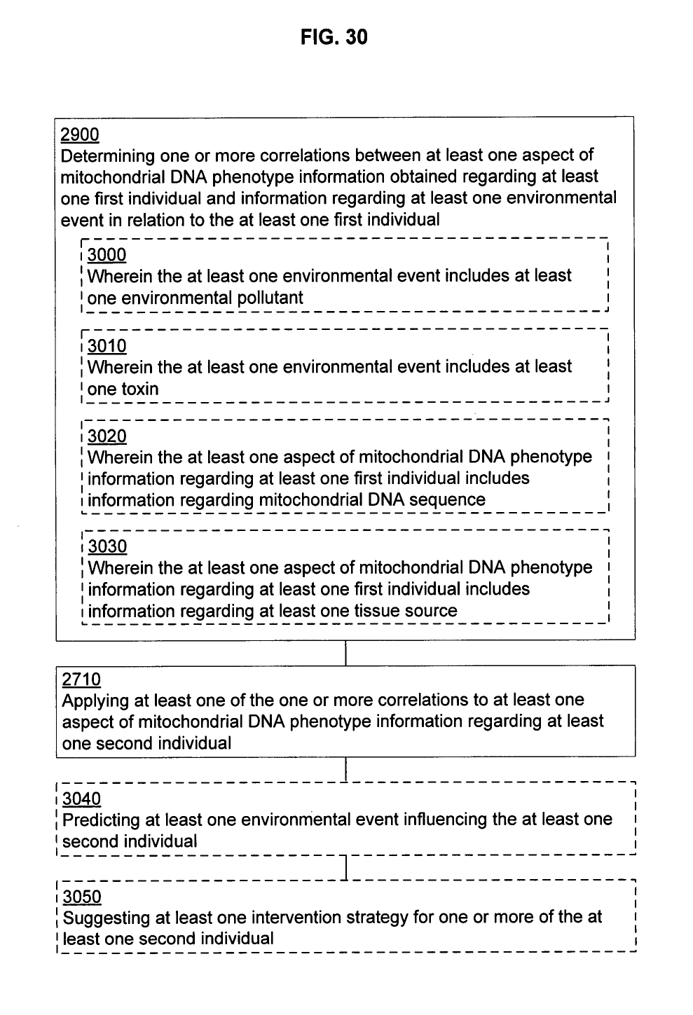

FIG. 30 illustrates further aspects of the method shown in FIG. 29. Block 2900 may include one or more of blocks 3000, 3010, 3020, or 3030. Block 3000 depicts wherein the at least on environmental event includes at least one environmental pollutant. Block 3010 shows wherein the at least one environmental event includes at least one toxin. Block 3020 illustrates wherein the at least one aspect of mitochondrial DNA phenotype information regarding at least one first individual includes information regarding mitochondrial DNA sequence. Block 3030 depicts wherein the at least one aspect of mitochondrial DNA phenotype information regarding at least one first individual includes information regarding at least one tissue source. The flowchart may also include at least one of blocks 3040 and 3050. Block 3040 shows predicting at least one environmental event influencing the at least one second individual. Block 3050 depicts suggesting at least one intervention strategy for one or more of the at least one second individual.

### Variation(s), or Implementation(s)

Those having skill in the art will recognize that the disclosure teaches modifications of the devices, structures, and/or processes within the spirit of the teaching herein. For example, methods and systems described herein may be beneficial for monitoring at least one aspect of mitochondrial DNA phenotype information of at least one individual during the course of therapeutic treatment with a therapeutic agent, investigational agent, energy-based therapy or surgical procedure, for example. As such, the at least one aspect of mitochondrial DNA phenotype information of at least one individual may be correlated with a positive or negative clinical outcome to a therapy and may be used to identify a patient population that will derive the most benefit from the therapy. For example, methods and systems described herein may be beneficial for correlating at least one aspect of mitochondrial DNA information regarding at least one individual with differential treatment response in a clinical or outpatient setting. The correlation between an aspect of mitochondrial DNA information regarding at least one individual with positive or negative clinical outcome to a therapy, such as a therapeutic agent, investigational agent or surgical procedure, for example, may be used to identify the patient population that will derive the most benefit from the therapy.

The at least one aspect of mitochondrial DNA phenotype information of at least one individual may be assessed and processed prior to initiation of treatment with a therapeutic agent, investigational agent, energy-based therapy or surgical procedure, for example, using the methods and systems described herein. As such, a diagnostic test or tests may be developed and used to determine the baseline of at least one aspect of mitochondrial DNA phenotype information of an individual prior to treatment or inclusion in a clinical trial (see e.g. Bai & Wong, Simultaneous detection and quantification of mitochondrial DNA deletion(s), depletion, and over-replication in patients with mitochondrial disease, J. Mol. Diagn. 7:613-622 (2005) and Liu et al., Rapid screening mitochondrial DNA mutation by using denaturing high-performance liquid chromatography, World J. Gastroenterol. 8:426-430 (2002), which are herein incorporated by reference). The baseline mitochondrial DNA phenotype information of a least one individual taken prior to treatment may be compared with known mitochondrial DNA phenotype information data collected, for example, from the scientific and medical literature and incorporated into the systems described herein. Known mitochondrial DNA phenotype information of at least one individual may be derived from individuals who have been previously treated with a particular therapeutic agent, investigational agent, energy-based therapy or surgical procedure and for whom data are available regarding positive and negative response to that particular therapy. As an example, sequence variations in the 12S and 16S rRNA of mitochondrial DNA predispose patients treated with aminoglycoside antibiotics to permanent hearing loss (see e.g. Hutchin, et al., A molecular basis for human hypersensitivity to aminoglycoside antibiotics, Nucleic Acids Res. 21:4174-4179 (1993), which is herein incorporated by reference). Data regarding, for example, positive and negative outcomes are routinely collected for each individual participating in a clinical trial under guidelines regulated by, for example, the Food and Drug Administration (FDA) (see e.g. Good Clinical Practice in FDA-Regulated Clinical Trials; available from the FDA). The methods and systems described herein may be used to derive correlations between baseline mitochondrial DNA phenotype information of a perspective patient and positive and negative outcomes of a particular therapy. As such, a physician or other medical practitioner may use these correlations, for example to guide prescribing practices or to establish inclusion/exclusion criteria for a clinical trial.

Data derived from the scientific and/or medical literature regarding known mitochondrial DNA phenotype information and incorporated into the methods and systems described herein may indicate a strong correlation between the baseline of at least one aspect of mitochondrial DNA phenotype information of at least one individual and one or more negative treatment outcome. As such, a diagnostic test or tests may be incorporated into the treatment regimen or clinical trial design to monitor the development and/or progression of negative outcomes correlated with individual-specific mitochondrial DNA phenotype information. For example, if increased blood pressure is correlated with specific mitochondrial DNA phenotype information and the therapy, routine use of a blood pressure monitor may be incorporated into the treatment regimen or clinical trial design. In some instances, a negative outcome of the therapy correlated with specific mitochondrial DNA phenotype information may be progressive. For instance, an individual having certain mitochondrial DNA phenotype information may be prone to liver damage as a result of therapy. Based on this information, for example, an individual may be excluded from the treatment regimen or clinical trial. Alternatively, a diagnostic test may be incorporated into the treatment regimen or clinical trial to routinely monitor, for example, liver enzymes as an indicator of potential liver damage. In some instances, a negative outcome of the therapy correlated with specific mitochondrial DNA phenotype information may be life-threatening, in which case an individual may be excluded from the treatment regimen or clinical trial.

The baseline of at least one aspect of mitochondrial DNA phenotype information of at least one individual may serve as a comparator for possible changes in mitochondrial DNA phenotype information noted through monitoring during the course of treatment and/or thereafter using the methods and systems described herein. As such, a diagnostic test or tests may be developed and used to periodically reassess the at least one aspect of mitochondrial DNA phenotype information of at least one individual during the course of treatment. Changes in the mitochondrial DNA phenotype information may be indicative of disease progression. A number of diseases have been linked to either mutations in human mitochondrial DNA or changes in the overall amount of human mitochondrial DNA. For example, mutations in human mitochondrial DNA increase with age and have been linked to various aspects of aging, such as sarcopenia or loss of muscle mass, Parkinson's and Alzheimer's diseases, ischemic heart disease, cataracts, and hearing loss, (see, e.g.: Kujoth et al., The role of mitochondrial DNA mutations in mammalian aging, PLOS Genetics, 3: e24 (2007); Wallace, Mitochondrial DNA sequence variation in human evolution and disease, PNAS USA 91:8739-8746 (1994); and Liu et al., Mutations in mitochondrial DNA accumulate differently in three different human tissues during aging, Nucleic Acids Research 26: 1268-1275 (1998), which are herein incorporated by reference). Changes in human mitochondrial DNA have also been associated with cancer and ulcerative colitis (see, e.g.: Chatterjee et al., Mitochondrial DNA mutations in human cancer, Oncogene, 25:4663-4674 (2006) and Fukushima & Fiocchi, Paradoxical decrease in mitochondrial DNA deletions in epithelial cells of active ulcerative colitis patients, Am. J. Physiol. Gastrointest. Liver Physiol. 283:G804-813 (2004), which are herein incorporated by reference).

Changes in the baseline of at least one aspect of mitochondrial DNA phenotype information monitored during the course of a treatment regimen or clinical trial using the methods and systems described herein may be indicative of a positive response to therapy and disease remission. For example, epithelial cells isolated from salivary rinses of patients with head and neck cancer have an increased mitochondrial DNA content relative to that of normal individuals (Jiang et al., Increased mitochondrial DNA content in saliva associated with head and neck cancer, Clin. Cancer Res. 11:2486-2491 (2005), which is herein incorporated by reference). In contrast, the mitochondrial DNA content of the cells in the salivary rinses of the cancer patients is significantly reduced following surgery and postoperative radiation therapy (Jiang et al., Decreased mitochondrial DNA content in posttreatment salivary rinses from head and neck cancer patients, Clin. Cancer Res. 12:1564-1569 (2006), which is herein incorporated by reference). In this instance, monitoring the aspects of mitochondrial DNA phenotype information before and after therapy may indicate how well the therapy has worked to ablate the cancerous cells.

The methods and systems described herein may also be beneficial in assessing the potential toxicity of an existing or investigational therapy in an outpatient setting or during clinical trial progression. For example, at least one aspect of the mitochondrial DNA phenotype information of an individual may be determined prior to initiation of a treatment and the data incorporated into a database accessible by the system described herein. As treatment proceeds, at least one aspect of mitochondrial DNA phenotype information may be periodically reassessed and monitored for any changes. Changes in aspects of the mitochondrial DNA phenotype information may or may not be correlated with one or more negative clinical outcome observed during the time course of the study. Any changes in aspects of the mitochondrial DNA information may be linked back to known information regarding specific mitochondrial DNA changes and possible outcomes. For example, nucleoside analogs used as antiviral therapy for human immunodeficiency virus (HIV) and hepatitis B are known to induce mutations in mitochondrial DNA as well as perturb mitochondrial DNA synthesis as measured by a decrease in overall mitochondrial DNA (see e.g. Martin, et al., Accumulation of mitochondrial DNA mutations in human immunodeficiency virus-infected patients treated with nucleoside analogue reverse-transcriptase inhibitors, Am. J. Hum. Genet. 72:549-560 (2003); and Johnson, et al., Toxicity of antiviral nucleoside analogs and the human mitochondrial DNA polymerase, J. Biol. Chem. 276:40847-40857 (2001), which are herein incorporated by reference). Several of the negative clinical outcomes associated with antiviral treatment, including lipoatrophy and neuropathy, mimic the symptoms associated with genetic mutations in mitochondrial DNA. As such, the FDA has recommended that all investigational antiviral agents be tested for effects on mitochondrial DNA (see e.g. Guidance for industry: antiviral product development—conducting and submitting virology studies to the agency, Food and Drug Administration, bearing the date of June 2006, which is herein incorporated by reference). As such, at least one aspect of the mitochondrial DNA phenotype information of at least one individual undergoing treatment with a nucleoside analog may be monitored using the methods and systems described herein to assess treatment associated toxicity.

In another example, treatment of human cells with X-ray irradiation induces a dose dependent increase in the number of mitochondrial DNA deletions (see, e.g.: Rogounovitch et al., Large deletion in mitochondrial DNA in radiation-associated human thyroid tumors, Cancer Res. 62:7031-7041 (2002), which is herein incorporated by reference). Similarly, treatment with the chemotherapy agents cisplatin and 5-fluorouracil may be associated with increased mitochondrial DNA damage in gastrointestinal epithelial cells (see, e.g.: Yanez et al., Chemotherapy induced gastrointestinal toxicity in rats: Involvement of mitochondrial DNA, gastrointestinal permeability and cyclooxygenase-2, J. Pharm. Pharmaceut. Sci. 6:308-314 (2003), which is herein incorporated by reference). As such, a physician or other medical practitioner may use the methods and systems described herein to assess and monitor the effects of radiotherapy and/or chemotherapy on at least one aspect of mitochondrial DNA phenotype information of at least one individual undergoing therapy. More generally, one or more changes in aspects of the mitochondrial DNA phenotype information over the course of treatment with, for example, an investigational agent, may indicate increased risk of developing a disease in the future. In addition, changes in aspects of the mitochondrial DNA phenotype information associated with a specific investigational agent can be compared, for example, with one or more agents with comparable method of action to determine if changes in at least one aspect of mitochondrial DNA phenotype information is a class phenomenon associated with all agents possessing a similar method of action or specific to the chemical entity under investigation.

The methods and systems described herein may be used, for example, in real time to generate correlations between at least one aspect of mitochondrial DNA phenotype information regarding at least one individual and positive or negative clinical outcomes observed during the course of a clinical trial. It is anticipated that the methods and systems described herein may be used in conjunction with clinical data management systems, computerized or otherwise, for monitoring clinical data as regulated by, for example, the FDA (see e.g. Guidance for Industry: Computerized Systems Used in Clinical Investigations, Federal Registrar, 72:26638, May 10, 2007, which is herein incorporated by reference). As the clinical trial progresses, certain aspects of mitochondrial DNA phenotype information may correlate very early with substantial benefit to one or more subpopulations of participating individuals. The clinical trial sponsor may choose to use these correlations to modify the clinical trial design by altering, for example, the inclusion/exclusion criteria for future enrollment of participants. Similarly, as the clinical trial progresses, certain aspects of mitochondrial DNA phenotype information may correlate with the occurrence of one or more negative clinical outcome that may require additional monitoring of specific individuals, an event not originally planned for in the clinical trial design. The correlations determined during the clinical trial between therapy and at least one aspect of mitochondrial DNA phenotype information and clinical outcome may be used, for example, to design additional clinical trials with a narrowed patient population for whom, for example, the therapy will have the most positive and least negative clinical outcome.

The methods and systems described herein may also be beneficial for predicting potential disease outcome and may aid a physician or other practitioner in developing appropriate treatment options. For example, a series of five primary mutations in mitochondrial DNA have been linked to Leber hereditary optic neuropathy (LHON), a maternally inherited form of late-onset vision loss (see, e.g.: Wallace, Mitochondrial DNA sequence variation in human evolution and disease, Proc. Natl. Acad. Sci. 91:8739-8746 (1994), which is herein incorporated by reference). Two of the most severe LHON mutations are also associated with pediatric neurodegenerative disease, suggesting that the milder LHON mutations may predispose individuals to late-onset neurodegenerative diseases, such as Parkinson's and Alzheimer's diseases. In this example, the physician or other practitioner may use at least one aspect of an individual's mitochondrial DNA phenotype information regarding LHON mutations to predict patient outcome and to determine appropriate treatment options, such as more frequent visits to the ophthalmologist and early tests for neurodegenerative disease.

In another example, a cytosine/adenine polymorphism in mitochondrial DNA at Mt5178 is associated with increased longevity in the Japanese population (see, e.g.: Kokaze, Genetic epidemiological studies of longevity-associated mitochondrial DNA 5178 C/A polymorphism, Environ. Health Prev. Med. 10:319-323 (2005), which is herein incorporated by reference). Differences are observed in blood pressure, serum lipid and protein levels, and intraocular pressure in Mt5178A versus Mt5178C individuals and these parameters are differentially affected by associated drinking and smoking habits. In this example, the physician or other practitioner may use at least one aspect of an individual's mitochondrial DNA phenotype information regarding, for example, the MtDNA5178 C/A polymorphism to predict patient outcome and to guide treatment options, such as more frequent blood pressure monitoring or discussions regarding moderation of lifestyle choices.

The methods and systems described herein may be beneficial in the field of assisted reproduction and may aid a physician or other practitioner in developing appropriate treatment options. For example, male fertility potential has been linked to the quality of the mitochondrial DNA in that increased deletions in sperm mitochondrial DNA leads to deficient oxidative phosphorylation, in turn causing abnormal metabolism and inadequate sperm motility (see, e.g.: Lewis et al., An algorithm for predict pregnancy in assisted reproduction, Human Reprod. 19:1385-1394 (2004), which is herein incorporated by reference). Similarly, oocytes with higher copy numbers of mitochondrial DNA are known to be associated with improved fertilization rates (see, e.g.: Spikings et al., Transmission of mitochondrial DNA following assisted reproduction and nuclear transfer, Human Reprod. 12:401-415 (2006), which is herein incorporated by reference). As such, the methods and systems described herein may be used to monitor at least one aspect of mitochondrial DNA phenotype information of at least one individual in the context, for example, of fertility treatment. In addition, the methods and systems described herein may be used to monitor at least one aspect of mitochondrial DNA phenotype information of at least one individual during the course of invasive oocyte reconstruction protocols. In general, mitochondrial DNA is maternally inherited and as such mitochondrial diseases are transmitted from mother to child. Invasive oocyte reconstruction protocols may involve either supplementing the oocyte of an older recipient with cytoplasm from a young donor or nuclear transfer of an oocyte nuclei from an older recipient into the enucleated oocyte of a young donor, in either case resulting in potential transfer of mitochondria and heteroplasmy (see, e.g.: Spikings et al., Transmission of mitochondrial DNA following assisted reproduction and nuclear transfer, Human Reprod. 12:401-415 (2006), which is herein incorporated by reference). As such, the methods and systems described herein may be used to monitor at least one aspect of mitochondrial DNA phenotype information of optionally the recipient, the donor, and/or the resulting off-spring to assess the degree of heteroplasmy and potential outcome.

The methods and systems described herein may be used as outlined above to develop a large body of correlative data regarding at least one aspect of mitochondrial DNA phenotype information of at least one individual and one or more medical therapies. In some embodiments, the at least one aspect of mitochondrial DNA phenotype information may include information from two or more individuals with a common attribute. At least one common attribute may include, for example, information regarding gender, height, weight, diabetes status, heart disease status, medical diagnosis, results on one or more medical tests, or ethnic background. Information regarding at least one tissue source may include information regarding the origin, storage, pathology, pathological subtype, or handling of the tissue and may include information regarding at least one neoplastic source, displastic source, diseased source, infectious source or cancerous source. At least one aspect of mitochondrial DNA phenotype information from a least one individual may be correlated, for example, with the MITOMAP database which is a compendium of polymorphisms and mutations of the human mitochondrial genome (see e.g. Ruiz-Pesini et al., An enhanced MITOMAP with a global mtDNA mutational phylogeny. Nucleic Acids Research 35 (Database issue):D823-D828. (2007), which is herein incorporated by reference). Similarly, at least one aspect of mitochondrial DNA phenotype information may be correlated with data retrieved, for example, from the scientific and medical literature (see e.g. Wong et al., Comprehensive scanning of the entire mitochondrial genome for mutations, Clin. Chem. 48:1901-1912 (2002), which is herein incorporated by reference). At least one aspect of mitochondrial DNA information may also be correlated with pharmacogenetic information, such as polymorphisms in genes encoding enzymes associated with drug metabolism and transport (see e.g. Goldstein, et al., Pharmacogenetics goes genomic, Nature Rev. Genet. 4:937-947 (2003), which is herein incorporated by reference). The accumulated correlation data may be beneficial not only for clinical trial design and progression as described herein, but also for prescribing practices following approval of a new therapy. For example, a physician or other practitioner may use the accumulated correlation data in combination with the mitochondrial DNA phenotype information of an individual to predict whether a specific medical therapy will provide a positive outcome. In addition, a physician or other practitioner may use the accumulated correlation data to predict specific negative outcomes that may be associated with a given treatment and at least one aspect of an individual's specific mitochondrial DNA phenotype information. As such, the physician or individual may use these data to assess the risk/benefit associated with a particular treatment option and make decisions regarding treatment accordingly. In the instance, for example, where there are multiple therapies of a given class available to treat a specific disease or condition, the physician or other practitioner may use the accumulated correlation data for each therapy in combination with at least one aspect of the individual's specific mitochondrial DNA phenotype information to choose the optimal treatment course.

The methods and systems described herein may be beneficial for monitoring changes in a least one aspect of mitochondrial DNA phenotype information of at least one individual in response to exposure to environmental toxins, for example, in the workplace. As such, changes in mitochondrial DNA phenotype information over time may be used, for example, to assess the precautions that have been put in place by the employer to protect the health and safety of employees and may guide decisions regarding the need for additional precautions. For example, the carcinogen asbestos was commonly used in automotive parts such as brake shoes, building materials such as insulation, floor and ceiling tiles, and wallboard, as well as in industrial materials such as fireproofing, insulation, and adhesives. While the current industrial use of asbestos has been curtailed, an employee may still be exposed to asbestos during the course of dismantling older materials. The most carcinogenic component of asbestos, crocidolite asbestos, causes damage to mitochondrial DNA at a concentration four-fold lower than that causing damage to nuclear DNA, suggesting that mitochondria are the initial target of asbestos-induced DNA damage and apoptosis (see, e.g.: Shukla et al., Asbestos induces mitochondrial DNA damage and dysfunction lined to the development of apoptosis, Am. J. Physiol. Lung Cell. Mol. Physiol. 285:L1018-L1025 (2003), which is herein incorporated by reference). As such, at least one aspect of mitochondrial DNA phenotype information of at least one individual exposed to asbestos in the workplace, for example, may be assessed at the initiation of employment and periodically reassessed and monitored using the methods and systems described herein to assess the potential health risks of asbestos exposure.

The methods and systems described herein may also be beneficial for monitoring changes in at least one aspect of mitochondrial DNA phenotype information of at least one individual in response to expected or unexpected exposure to environmental toxins. Unexpected exposure may constitute a single event, such as an accidental release of radiation or of a toxic gas or a liquid. Alternatively, an expected or unexpected exposure may represent long term exposure to a toxin in the soil, water and/or air, or associated, for example, with a residence. As such, the expected or unexpected exposure may effect a single individual or an entire community. As an example, the deleterious health effects of smoking cigarettes are well known, including decline in pulmonary function and increased risk of pulmonary diseases such as chronic obstructive pulmonary disease, emphysema, and lung cancer. Specific deletions in mitochondrial DNA are observed in lung and plasma samples of cigarette smokers and increase proportionally with the smoking index or number of pack-years (see, e.g.: Fahn et al., Smoking associated mitochondrial DNA mutations and lipid peroxidation in human lung tissues, Am. J. Respir. Cell Mol. Biol. 19:901-919 (1998), which is herein incorporated by reference). In addition, pack-years of cigarette smoking is positively correlated with increased overall mitochondrial DNA content relative to that of non-smokers (see, e.g.: Masayesva et al., Mitochondrial DNA content increases in response to cigarette smoking, Cancer Epidemiol. Biomarkers Prev. 15:19-25 (2006), which is herein incorporated by reference). Lastly, exposure to second hand smoke is also correlated with increased mitochondrial DNA damage (see, e.g.: Knight-Lozano et al., Cigarette smoke exposure and hypercholesterolemia increase mitochondrial damage in cardiovascular tissue, Circ. 105:849-854 (2002), which is herein incorporated by reference). As such, at least one aspect of mitochondrial DNA phenotype information of at least one individual exposed to cigarette smoke either directly or indirectly in a residence or workplace, for example, may be monitored using the methods and systems described herein to assess the potential health risks of exposure to cigarette smoke.

As another example, a community may experience long term exposure to drinking water contaminated with arsenic, leading to an increased risk of dermal lesions, peripheral neuropathy, skin, bladder and lung cancers and peripheral vascular disease (see, e.g.: World Health Organization, Arsenic in drinking water: Background document for development of WHO Guidelines for Drinking Water Quality; WHO/SDS/WSH/03.04/75 (2003), which is herein incorporated by reference). Chronic exposure of cells to inorganic arsenic results in reduced mitochondrial DNA copy number and a heterogeneous increase in large heteroplasmic mitochondrial DNA deletions (see, e.g.: Partridge et al., Arsenic induced mitochondrial DNA damage and altered mitochondrial oxidative function: Implications for genotoxic mechanisms in mammalian cells, Cancer Res. 67:5239-5247 (2007), which is herein incorporated by reference). As such, at least one aspect of mitochondrial DNA phenotype information of at least one individual in the community may be monitored using the methods and systems described herein to assess, for example, the effect of long-term arsenic exposure.

The methods and systems described herein may be used as outlined above to develop a large body of correlative data regarding at least one aspect of mitochondrial DNA phenotype information of at least one individual and one or more environmental toxins. In some embodiments, the at least one aspect of mitochondrial DNA phenotype information may include information from two or more individuals with a common attribute. At least one common attribute may include, for example, information regarding gender, height, weight, ethnic background, occupation, residence, lifestyle choices, and/or health status. At least one aspect of mitochondrial DNA phenotype information from a least one individual may be correlated, for example, with the MITOMAP database which is a compendium of polymorphisms and mutations of the human mitochondrial genome (see e.g. Ruiz-Pesini et al., An enhanced MITOMAP with a global mtDNA mutational phylogeny. Nucleic Acids Research 35 (Database issue):D823-D828. (2007), which is herein incorporated by reference). At least one aspect of mitochondrial DNA phenotype information may also be correlated with information regarding specific environmental toxins and health hazards available, for example, from the Department of Health and Human Services, Agency for Toxic Substances & Disease Registry (ATSDR). The accumulated data may be beneficial not only for monitoring mitochondrial DNA phenotype information associated with potential health risks of known environmental toxins, but also for identifying new toxins.

Other modifications of the subject matter herein will be appreciated by one of ordinary skill in the art in light of the teachings herein.

Those having skill in the art will recognize that the state of the art has progressed to the point where there is little distinction left between hardware and software implementations of aspects of systems; the use of hardware or software is generally (but not always, in that in certain contexts the choice between hardware and software can become significant) a design choice representing cost vs. efficiency tradeoffs. Those having skill in the art will appreciate that there are various vehicles by which processes or systems or other technologies described herein can be effected (e.g., hardware, software, or firmware), and that the preferred vehicle will vary with the context in which the processes or systems or other technologies are deployed. For example, if an implementer determines that speed and accuracy are paramount, the implementer may opt for a mainly hardware or firmware vehicle; alternatively, if flexibility is paramount, the implementer may opt for a mainly software implementation; or, yet again alternatively, the implementer may opt for some combination of hardware, software, or firmware. Hence, there are several possible vehicles by which the processes or devices or other technologies described herein may be effected, none of which is inherently superior to the other in that any vehicle to be utilized is a choice dependent upon the context in which the vehicle will be deployed and the specific concerns (e.g., speed, flexibility, or predictability) of the implementer, any of which may vary. Those skilled in the art will recognize that optical aspects of implementations will typically employ optically-oriented hardware, software, and or firmware.

In a general sense, those skilled in the art will recognize that the various aspects described herein which can be implemented, individually or collectively, by a wide range of hardware, software, firmware, or any combination thereof can be viewed as being composed of various types of “electrical circuitry.” Consequently, as used herein “electrical circuitry” includes, but is not limited to, electrical circuitry having at least one discrete electrical circuit, electrical circuitry having at least one integrated circuit, electrical circuitry having at least one application specific integrated circuit, electrical circuitry forming a general purpose computing device configured by a computer program (e.g., a general purpose computer configured by a computer program which at least partially carries out processes or devices described herein, or a microprocessor configured by a computer program which at least partially carries out processes or devices described herein), electrical circuitry forming a memory device (e.g., forms of random access memory), or electrical circuitry forming a communications device (e.g., a modem, communications switch, or optical-electrical equipment). Those having skill in the art will recognize that the subject matter described herein may be implemented in an analog or digital fashion or some combination thereof.

One skilled in the art will recognize that the herein described components (e.g., steps), devices, and objects and the discussion accompanying them are used as examples for the sake of conceptual clarity and that various configuration modifications are within the skill of those in the art. Consequently, as used herein, the specific exemplars set forth and the accompanying discussion are intended to be representative of their more general classes. In general, use of any specific exemplar herein is also intended to be representative of its class, and the non-inclusion of such specific components (e.g., steps), devices, and objects herein should not be taken as indicating that limitation is desired.

The herein described subject matter sometimes illustrates different components contained within, or connected with, different other components. It is to be understood that such depicted architectures are merely exemplary, and that in fact many other architectures can be implemented which achieve the same functionality. In a conceptual sense, any arrangement of components to achieve the same functionality is effectively “associated” such that the desired functionality is achieved. Hence, any two components herein combined to achieve a particular functionality can be seen as “associated with” each other such that the desired functionality is achieved, irrespective of architectures or intermedial components. Likewise, any two components so associated can also be viewed as being “operably connected,” or “operably coupled,” to each other to achieve the desired functionality, and any two components capable of being so associated can also be viewed as being “operably couplable,” to each other to achieve the desired functionality. Specific examples of operably couplable include but are not limited to physically mateable or physically interacting components or wirelessly interactable or wirelessly interacting components or logically interacting or logically interactable components.

While particular aspects of the present subject matter described herein have been shown and described, it will be apparent to those skilled in the art that, based upon the teachings herein, changes and modifications may be made without departing from the subject matter described herein and its broader aspects and, therefore, the appended claims are to encompass within their scope all such changes and modifications as are within the true spirit and scope of the subject matter described herein. Furthermore, it is to be understood that the invention is defined by the appended claims. It will be understood by those within the art that, in general, terms used herein, and especially in the appended claims (e.g., bodies of the appended claims) are generally intended as “open” terms (e.g., the term “including” should be interpreted as “including but not limited to,” the term “having” should be interpreted as “having at least,” the term “includes” should be interpreted as “includes but is not limited to,” etc.). It will be further understood by those within the art that if a specific number of an introduced claim recitation is intended, such an intent will be explicitly recited in the claim, and in the absence of such recitation no such intent is present. For example, as an aid to understanding, the following appended claims may contain usage of the introductory phrases “at least one” and “one or more” to introduce claim recitations. However, the use of such phrases should not be construed to imply that the introduction of a claim recitation by the indefinite articles “a” or “an” limits any particular claim containing such introduced claim recitation to inventions containing only one such recitation, even when the same claim includes the introductory phrases “one or more” or “at least one” and indefinite articles such as “a” or “an” (e.g., “a” or “an” should typically be interpreted to mean “at least one” or “one or more”); the same holds true for the use of definite articles used to introduce claim recitations. In addition, even if a specific number of an introduced claim recitation is explicitly recited, those skilled in the art will recognize that such recitation should typically be interpreted to mean at least the recited number (e.g., the bare recitation of “two recitations,” without other modifiers, typically means at least two recitations, or two or more recitations). Furthermore, in those instances where a convention analogous to “at least one of A, B, and C, etc.” is used, in general such a construction is intended in the sense one having skill in the art would understand the convention (e.g., “a system having at least one of A, B, and C” would include but not be limited to systems that have A alone, B alone, C alone, A and B together, A and C together, B and C together, or A, B, and C together, etc.). In those instances where a convention analogous to “at least one of A, B, or C, etc.” is used, in general such a construction is intended in the sense one having skill in the art would understand the convention (e.g., “a system having at least one of A, B, or C” would include but not be limited to systems that have A alone, B alone, C alone, A and B together, A and C together, B and C together, or A, B, and C together, etc.). It will be further understood by those within the art that virtually any disjunctive word or phrase presenting two or more alternative terms, whether in the description, claims, or drawings, should be understood to contemplate the possibilities of including one of the terms, either of the terms, or both terms. For example, the phrase “A or B” will be understood to include the possibilities of “A” or “B” or “A and B.”

With respect to the appended claims, those skilled in the art will appreciate that recited operations therein may generally be performed in any order. Examples of such alternate orderings may include overlapping, interleaved, interrupted, reordered, incremental, preparatory, supplemental, simultaneous, reverse, or other variant orderings, unless context dictates otherwise. With respect to context, even terms like “responsive to,” “related to,” or other past-tense adjectives are generally not intended to exclude such variants, unless context dictates otherwise.

With respect to the use of substantially any plural or singular terms herein, those having skill in the art can translate from the plural to the singular or from the singular to the plural as is appropriate to the context or application. The various singular/plural permutations are not expressly set forth herein for sake of clarity.

All of the above U.S. patents, U.S. patent application publications, U.S. patent applications, foreign patents, foreign patent applications and non-patent publications referred to in this specification or listed in any Application Data Sheet, are incorporated herein by reference, to the extent not inconsistent herewith.

The foregoing detailed description has set forth various embodiments of the devices or processes via the use of block diagrams, flowcharts, or examples. Insofar as such block diagrams, flowcharts, or examples contain one or more functions or operations, it will be understood by those within the art that each function or operation within such block diagrams, flowcharts, or examples can be implemented, individually or collectively, by a wide range of hardware, software, firmware, or virtually any combination thereof. In one embodiment, several portions of the subject matter described herein may be implemented via Application Specific Integrated Circuits (ASICs), Field Programmable Gate Arrays (FPGAs), digital signal processors (DSPs), or other integrated formats. However, those skilled in the art will recognize that some aspects of the embodiments disclosed herein, in whole or in part, can be equivalently implemented in integrated circuits, as one or more computer programs running on one or more computers (e.g., as one or more programs running on one or more computer systems), as one or more programs running on one or more processors (e.g., as one or more programs running on one or more microprocessors), as firmware, or as virtually any combination thereof, and that designing the circuitry or writing the code for the software and or firmware would be well within the skill of one of skill in the art in light of this disclosure. In addition, those skilled in the art will appreciate that the mechanisms of the subject matter described herein are capable of being distributed as a program product in a variety of forms, and that an illustrative embodiment of the subject matter described herein applies regardless of the particular type of signal bearing medium used to actually carry out the distribution. Examples of a signal bearing medium include, but are not limited to, the following: a recordable type medium such as a floppy disk, a hard disk drive, a Compact Disc (CD), a Digital Video Disk (DVD), a digital tape, a computer memory, etc.; and a transmission type medium such as a digital or an analog communication medium (e.g., a fiber optic cable, a waveguide, a wired communications link, a wireless communication link, etc.).

The various aspects and embodiments disclosed herein are for purposes of illustration and are not intended to be limiting, with the true scope and spirit being indicated by the following claims.

What is claimed is:

1. A method comprising:
determining a similarity or a dissimilarity between at least one aspect of mitochondrial DNA phenotype information regarding at least one first individual and at least one aspect of mitochondrial DNA phenotype information regarding at least one second individual, wherein the at least one second individual had been influenced by at least one mitochondrial DNA-influencing event.

2. (canceled)

3. The method of claim 1, wherein the at least one aspect of mitochondrial DNA phenotype information regarding at least one first individual includes information regarding mitochondrial DNA sequence.

4. The method of claim 1, wherein the at least one aspect of mitochondrial DNA phenotype information regarding at least one first individual includes information regarding heteroplasmy.

5. The method of claim 1, wherein the at least one aspect of mitochondrial DNA phenotype information regarding at least one first individual includes information regarding one or more maternal relatives of the at least one first individual.

6. The method of claim 1, wherein the at least one aspect of mitochondrial DNA phenotype information regarding at least one first individual includes information regarding mosaicism of the at least one first individual.

7.-9. (canceled)

10. The method of claim 1, wherein the at least one mitochondrial DNA-influencing event includes at least one medical therapy.

11. The method of claim 1, wherein the at least one mitochondrial DNA-influencing event includes at least one environmental event.

12. The method of claim 1, further comprising:
one or more instructions for correlating the at least one aspect of mitochondrial DNA phenotype information regarding at least one second individual with previous mitochondrial DNA information regarding the at least one second individual, wherein the previous mitochondrial DNA information was ascertained prior to the mitochondrial DNA-influencing event.

13. (canceled)

14. The method of claim 1, further comprising:
one or more instructions for suggesting at least one intervention strategy for the at least one first individual in reference to the at least one correlation.

15. A system comprising:
at least one computer program for use with at least one computer system and wherein the computer program includes a plurality of instructions, including but not limited to:
one or more instructions for determining at least one correlation between at least one mitochondrial DNA-influencing event and at least one aspect of mitochondrial DNA phenotype information regarding at least one individual.

16. (canceled)

17. The system of claim 15, wherein the at least one aspect of mitochondrial DNA phenotype information regarding at least one individual includes information regarding mitochondrial DNA sequence.

18. The system of claim 15, wherein the at least one aspect of mitochondrial DNA phenotype information regarding at least one individual includes information regarding heteroplasmy.

19. The system of claim 15, wherein the at least one aspect of mitochondrial DNA phenotype information regarding an individual includes information regarding one or more maternal relatives of the at least one individual.

20. The system of claim 15, wherein the at least one aspect of mitochondrial DNA phenotype information regarding at least one individual includes information regarding mosaicism of at least one individual.

21.-23. (canceled)

24. The system of claim 15, wherein the at least one mitochondrial DNA-influencing event includes at least one medical therapy.

25. The system of claim 15, wherein the at least one mitochondrial DNA-influencing event includes at least one environmental event.

26. The system of claim 15, further comprising:
one or more instructions for correlating the at least one aspect of mitochondrial DNA phenotype information regarding at least one individual with previous mitochondrial DNA information regarding the at least one individual, wherein the previous mitochondrial DNA information was ascertained prior to the mitochondrial DNA-influencing event.

27. (canceled)

28. The system of claim 15, further comprising:
one or more instructions for suggesting at least one intervention strategy in reference to the at least one correlation.

29. A system comprising:
at least one computer program for use with at least one computer system and wherein the computer program includes a plurality of instructions, including but not limited to:
one or more instructions for determining a similarity or a dissimilarity between at least one aspect of mitochondrial DNA phenotype information regarding at least one first individual and at least one aspect of mitochondrial DNA phenotype information regarding at least one second individual, wherein the at least one second individual had been influenced by at least one mitochondrial DNA-influencing event.

30. (canceled)

31. The system of claim 29, wherein the at least one aspect of mitochondrial DNA phenotype information regarding at least one first individual includes information regarding mitochondrial DNA sequence.

32. The system of claim 29, wherein the at least one aspect of mitochondrial DNA phenotype information regarding at least one first individual includes information regarding heteroplasmy.

33. The system of claim 29, wherein the at least one aspect of mitochondrial DNA phenotype information regarding at least one first individual includes information regarding one or more maternal relatives of the at least one first individual.

34. The system of claim 29, wherein the at least one aspect of mitochondrial DNA phenotype information regarding at least one first individual includes information regarding mosaicism of the at least one first individual.

35.-37. (canceled)

38. The system of claim 29, wherein the at least one mitochondrial DNA-influencing event includes at least one medical therapy.

39. The system of claim 29, wherein the at least one mitochondrial DNA-influencing event includes at least one environmental event.

40. The system of claim 29, further comprising:
one or more instructions for correlating the at least one aspect of mitochondrial DNA phenotype information regarding at least one second individual with previous mitochondrial DNA information regarding the at least one second individual, wherein the previous mitochondrial DNA information was ascertained prior to the mitochondrial DNA-influencing event.

41. (canceled)

42. The system of claim 29, further comprising:
one or more instructions for suggesting at least one intervention strategy for the at least one first individual in reference to the at least one correlation.

43. A system comprising:
at least one computer program for use with at least one computer system and wherein the computer program includes a plurality of instructions, including but not limited to:
one or more instructions for determining one or more correlations between at least one aspect of mitochondrial DNA phenotype information obtained regarding at least one first individual and information regarding at least one mitochondrial DNA-influencing event in relation to the at least one first individual; and
one or more instructions for applying at least one of the one or more correlations to at least one aspect of mitochondrial DNA phenotype information regarding at least one second individual.

44. The system of claim 43, wherein the at least one mitochondrial DNA-influencing event includes at least one medical therapy.

45. The system of claim 43, wherein the at least one mitochondrial DNA-influencing event includes at least one environmental event.

46. The system of claim 43, wherein the at least one aspect of mitochondrial DNA phenotype information regarding at least one first individual includes information regarding mitochondrial DNA sequence.

47. (canceled)

48. The system of claim 43, further comprising:
one or more instructions for predicting at least one mitochondrial DNA-influencing event influencing the at least one second individual.

49. The system of claim 43, further comprising:
one or more instructions for suggesting at least one intervention strategy for one or more of the at least one second individual.

50. A system comprising:
at least one computer program for use with at least one computer system and wherein the computer program includes a plurality of instructions, including but not limited to:
one or more instructions for determining one or more correlations between at least one aspect of mitochondrial DNA phenotype information obtained regarding at least one first individual and information regarding at least one medical therapy in relation to the at least one first individual; and
one or more instructions for applying at least one of the one or more correlations to at least one aspect of mitochondrial DNA phenotype information regarding at least one second individual.

51. The system of claim 50, wherein the at least one medical therapy includes at least one therapeutic agent.

52. The system of claim 50, wherein the at least one medical therapy includes at least one energy-based procedure.

53. The system of claim 50, wherein the at least one aspect of mitochondrial DNA phenotype information regarding at least one first individual includes information regarding mitochondrial DNA sequence.

54. (canceled)

55. The system of claim 50, further comprising:
one or more instructions for suggesting at least one intervention strategy for one or more of the at least one second individual.

56. A system comprising:
at least one computer program for use with at least one computer system and wherein the computer program includes a plurality of instructions, including but not limited to:
one or more instructions for determining one or more correlations between at least one aspect of mitochondrial DNA phenotype information obtained regarding at least one first individual and information regarding at least one environmental event in relation to the at least one first individual; and
one or more instructions for applying at least one of the one or more correlations to at least one aspect of mitochondrial DNA phenotype information regarding at least one second individual.

57. The system of claim 56, wherein the at least one environmental event includes at least one environmental pollutant.

58. The system of claim 56, wherein the at least one environmental event includes at least one toxin.

59. The system of claim 56, wherein the at least one aspect of mitochondrial DNA phenotype information regarding at least one first individual includes information regarding mitochondrial DNA sequence.

60. (canceled)

61. The system of claim 56, further comprising:
one or more instructions for predicting at least one environmental event influencing the at least one second individual.

62. The system of claim 56, further comprising:
one or more instructions for suggesting at least one intervention strategy for one or more of the at least one second individual.
